(19) United States
(12) Patent Application Publication
(10) Pub. No.: US 2024/0327515 A1
(43) Pub. Date: Oct. 3, 2024

(54) ANTI-CD3 ANTIBODIES

(71) Applicant: iBio, Inc., Bryan, TX (US)

(72) Inventors: Matthew P. Greving, Rancho Santa Fe, CA (US); Alexander Tomoaki Taguchi, San Diego, CA (US); Cody Allen Moore, San Diego, CA (US); Hari Hara S. Potula, Freemont, CA (US); Gao Liu, Brisbane, CA (US)

(21) Appl. No.: 18/619,648

(22) Filed: Mar. 28, 2024

Related U.S. Application Data
(60) Provisional application No. 63/456,240, filed on Mar. 31, 2023.

(51) Int. Cl. C07K 16/28

(52) U.S. Cl. CPC C07K 16/2809; C07K 2317/24; C07K 2317/31; C07K 2317/35; C07K 2317/622; C07K 2317/626

(57) ABSTRACT

Provided herein are novel human anti-CD3 antigen-binding polypeptides, treatments, nucleic acids, vectors, their preparation and their use in the treatment and/or diagnosis of various diseases, and also relates to bispecific antibody molecules capable of activating immune effector cells and their use in diagnosis and/or treatment of various diseases.

### CROSS-REFERENCE TO RELATED APPLICATIONS

This application claims priority to U.S. Provisional Application Ser. No. 63/456,240, filed Mar. 31, 2023, and, the entire contents of which are incorporated herein by reference.

### TECHNICAL FIELD OF THE INVENTION

This document relates to materials and methods for triggering an immune response, and particularly to the use of novel anti-CD3 antibodies to trigger immune activation or tolerance, and to treat autoimmune diseases, cancer, antimicrobial, antiviral, and other immune responses.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Mar. 27, 2024, is named “IBIO1040.xml” and is 2,893,154 bytes in size. The sequence listing contained in this .XML file is part of the specification and is hereby incorporated by reference herein in its entirety.

### BACKGROUND OF THE INVENTION

Without limiting the scope of the invention, its background is described in connection with anti-CD3 antibodies.

Such antibodies are taught by Jishage, et al., in U.S. Pat. No. 11,612,149, entitled, “Non-human Animal Having Human CD3 Gene Substituted For Endogenous CD3 Gene”. These inventors are said to teach genetically modified non-human animals that are deficient in at least one or more types of CD3 genes selected from the group consisting of endogenous CD3ε, CD3δ, and CD3γ in its genome and then functionally express at least one or more types of human CD3 genes selected from the group consisting of human CD3ε, CD3δ, and CD3γ. The genetically modified non-human animals are said to provide mature T cell differentiation and production, and immunocompetent cells including T cells.

Additional antibodies are taught by Van Den Brink, et al., in U.S. Pat. No. 11,613,575, entitled, “Humanized Or Chimeric CD3 Antibodies”. These inventors are said to teach humanized or chimeric antibodies binding CD3, bispecific antibodies, compositions, pharmaceutical compositions, use of the antibodies in the treatment of a disease, and method of treatment.

Finally, other antibodies are taught by Granada, et al., in U.S. Patent Publication No 20230037682, entitled, “BINDING MOLECULES AGAINST CD3 AND USES THEREOF”. These applicants are said to teach binding molecules that specifically bind to CD3, such as monospecific binding molecules that specifically bind to CD3 and multi-specific binding molecules (MBMs) that specifically bind to CD3 and a tumor-associated antigen, conjugates comprising the CD3 binding molecules, and pharmaceutical compositions comprising the CD3 binding molecules and conjugates. They are further said to teach methods of using the CD3 binding molecules, conjugates, and pharmaceutical compositions to activate T cells in a subject, for example a subject having a cancer or autoimmune disease, recombinant host cells engineered, and methods of producing the CD3 binding molecules by culturing the host cells under conditions in which the CD3 binding molecules are expressed.

Despite these advances, novel anti-CD3 antibodies are needed that have unique binding characteristics, target specific epitopes, and can be used for treating cancer or autoimmune diseases.

### SUMMARY OF THE INVENTION

As embodied and broadly described herein, an aspect of the present disclosure relates to an anti-CD3 antibody, wherein the antibody comprises: a heavy chain variable domain (VH) complementarity determining region 1 (CDR1) comprising the amino acid sequence of any one of the following SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, 801, 811, 821, 831, 841, 851, 861, 871, 881, 891, 901, 911, 921, 931, 941, 951, 961, 971, 981, 991, 1001, 1011, 1021, 1031, 1041, 1051, 1061, 1071, 1081, 1091, 1101, 1111, 1121, 1131, 1141, 1151, 1161, 1171, 1181, 1191, 1201, 1211, 1221, 1231, 1241, 1251, 1261, 1271, 1281, 1291, 1301, 1311, 1321, 1331, 1341, 1351, 1361, 1371, 1381, 1391, 1401, 1411, 1421, 1431, 1441, 1451, 1461, 1471, 1481, 1491, 1501, 1511, 1521, 1531, 1541, 1551, 1561, 1571, 1581, 1591, 1601, 1611, 1621, 1631, 1641, 1651, 1661, 1671, 1681, 1691, 1701, 1711, 1721, 1731, 1741, 1751, 1761, 1771, 1781, 1791, 1801, 1811, 1821, 1831, 1841, 1851, 1861, 1871, 1881, 1891, 1901, 1911, 1916, 1921, 1926, 1931, 1936, 1941, 1946, 1951, 1956, 1961, 1966, 1971, 1976, 1981, 1986, 1991, 1996, 2001, 2006, 2011, 2016, 2021, 2026, 2031, 2036, 2041, 2046, 2051, 2056, 2061, 2066, 2071, 2076, 2081, 2086, 2091, 2096, 2101, 2106, 2111, 2116, 2121, 2126, 2131, 2136, 2141, 2146, 2151, 2156, 2161, 2166, 2171, 2176, 2181, or 2186; and a VH CDR2 comprising the amino acid sequence of any one of the following SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, 792, 802, 812, 822, 832, 842, 852, 862, 872, 882, 892, 902, 912, 922, 932, 942, 952, 962, 972, 982, 992, 1002, 1012, 1022, 1032, 1042, 1052, 1062, 1072, 1082, 1092, 1102, 1112, 1122, 1132, 1142, 1152, 1162, 1172, 1182, 1192, 1202, 1212, 1222, 1232, 1242, 1252, 1262, 1272, 1282, 1292, 1302, 1312, 1322, 1332, 1342, 1352, 1362, 1372, 1382, 1392, 1402, 1412, 1422, 1432, 1442, 1452, 1462, 1472, 1482, 1492, 1502, 1512, 1522, 1532, 1542, 1552, 1562, 1572, 1582, 1592, 1602, 1612, 1622, 1632, 1642, 1652, 1662, 1672, 1682, 1692, 1702, 1712, 1722, 1732, 1742, 1752, 1762, 1772, 1782, 1792, 1802, 1812, 1822, 1832, 1842, 1852, 1862, 1872, 1882, 1892, 1902, 1912, 1917, 1922, 1927, 1932, 1937, 1942, 1947, 1952, 1957, 1962, 1967, 1972, 1977, 1982, 1987, 1992, 1997, 2002, 2007, 2012, 2017, 2022, 2027, 2032, 2037, 2042, 2047, 2052, 2057, 2062, 2067, 2072, 2077, 2082, 2087, 2092, 2097, 2102, 2107, 2112, 2117, 2122, 2127, 2132, 2137, 2142, 2147, 2152, 2157, 2162, 2167, 2172, 2177, 2182, or 2187; and a VH CDR3 comprising the amino acid sequence of any one of the following SEQ ID NOs: 3, 13, 23, 33, 43, 53, 63, 73, 83, 93, 103, 113, 123, 133, 143, 153, 163, 173, 183, 193, 203, 213, 223, 233, 243, 253, 263, 273, 283, 293, 303, 313, 323, 333, 343, 353, 363, 373, 383, 393, 403, 413, 423, 433, 443, 453, 463, 473, 483, 493, 503, 513, 523, 533, 543, 553, 563, 573, 583, 593, 603, 613, 623, 633, 643, 653, 663, 673, 683, 693, 703, 713, 723, 733, 743, 753, 763, 773, 783, 793, 803, 813, 823, 833, 843, 853, 863, 873, 883, 893, 903, 913, 923, 933, 943, 953, 963, 973, 983, 993, 1003, 1013, 1023, 1033, 1043, 1053, 1063, 1073, 1083, 1093, 1103, 1113, 1123, 1133, 1143, 1153, 1163, 1173, 1183, 1193, 1203, 1213, 1223, 1233, 1243, 1253, 1263, 1273, 1283, 1293, 1303, 1313, 1323, 1333, 1343, 1353, 1363, 1373, 1383, 1393, 1403, 1413, 1423, 1433, 1443, 1453, 1463, 1473, 1483, 1493, 1503, 1513, 1523, 1533, 1543, 1553, 1563, 1573, 1583, 1593, 1603, 1613, 1623, 1633, 1643, 1653, 1663, 1673, 1683, 1693, 1703, 1713, 1723, 1733, 1743, 1753, 1763, 1773, 1783, 1793, 1803, 1813, 1823, 1833, 1843, 1853, 1863, 1873, 1883, 1893, 1903, 1913, 1918, 1923, 1928, 1933, 1938, 1943, 1948, 1953, 1958, 1963, 1968, 1973, 1978, 1983, 1988, 1993, 1998, 2003, 2008, 2013, 2018, 2023, 2028, 2033, 2038, 2043, 2048, 2053, 2058, 2063, 2068, 2073, 2078, 2083, 2088, 2093, 2098, 2103, 2108, 2113, 2118, 2123, 2128, 2133, 2138, 2143, 2148, 2153, 2158, 2163, 2168, 2173, 2178, 2183, or 2188; and a light chain variable domain (VL) CDR1 comprising the amino acid sequence of any one of the following SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, 796, 806, 816, 826, 836, 846, 856, 866, 876, 886, 896, 906, 916, 926, 936, 946, 956, 966, 976, 986, 996, 1006, 1016, 1026, 1036, 1046, 1056, 1066, 1076, 1086, 1096, 1106, 1116, 1126, 1136, 1146, 1156, 1166, 1176, 1186, 1196, 1206, 1216, 1226, 1236, 1246, 1256, 1266, 1276, 1286, 1296, 1306, 1316, 1326, 1336, 1346, 1356, 1366, 1376, 1386, 1396, 1406, 1416, 1426, 1436, 1446, 1456, 1466, 1476, 1486, 1496, 1506, 1516, 1526, 1536, 1546, 1556, 1566, 1576, 1586, 1596, 1606, 1616, 1626, 1636, 1646, 1656, 1666, 1676, 1686, 1696, 1706, 1716, 1726, 1736, 1746, 1756, 1766, 1776, 1786, 1796, 1806, 1816, 1826, 1836, 1846, 1856, 1866, 1876, 1886, 1896, 1906, 2161, 2166, 2171, 2176, 2181, 2186, 2191, 2196, 2201, 2206, 2211, 2216, 2221, 2226, 2231, 2236, 2241, 2246, 2251, 2256, 2261, 2266, 2271, 2276, 2281, 2286, 2291, 2296, 2301, 2306, 2311, 2316, 2321, 2326, 2331, or 2336; and a VL CDR2 comprising the amino acid sequence of any one of the following SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, 797, 807, 817, 827, 837, 847, 857, 867, 877, 887, 897, 907, 917, 927, 937, 947, 957, 967, 977, 987, 997, 1007, 1017, 1027, 1037, 1047, 1057, 1067, 1077, 1087, 1097, 1107, 1117, 1127, 1137, 1147, 1157, 1167, 1177, 1187, 1197, 1207, 1217, 1227, 1237, 1247, 1257, 1267, 1277, 1287, 1297, 1307, 1317, 1327, 1337, 1347, 1357, 1367, 1377, 1387, 1397, 1407, 1417, 1427, 1437, 1447, 1457, 1467, 1477, 1487, 1497, 1507, 1517, 1527, 1537, 1547, 1557, 1567, 1577, 1587, 1597, 1607, 1617, 1627, 1637, 1647, 1657, 1667, 1677, 1687, 1697, 1707, 1717, 1727, 1737, 1747, 1757, 1767, 1777, 1787, 1797, 1807, 1817, 1827, 1837, 1847, 1857, 1867, 1877, 1887, 1897, 1907, 2162, 2167, 2172, 2177, 2182, 2187, 2192, 2197, 2202, 2207, 2212, 2217, 2222, 2227, 2232, 2237, 2242, 2247, 2252, 2257, 2262, 2267, 2272, 2277, 2282, 2287, 2292, 2297, 2302, 2307, 2312, 2317, 2322, 2327, 2332, or 2337; and a VL CDR3 comprising the amino acid sequence of any one of the following SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, 528, 538, 548, 558, 568, 578, 588, 598, 608, 618, 628, 638, 648, 658, 668, 678, 688, 698, 708, 718, 728, 738, 748, 758, 768, 778, 788, 798, 808, 818, 828, 838, 848, 858, 868, 878, 888, 898, 908, 918, 928, 938, 948, 958, 968, 978, 988, 998, 1008, 1018, 1028, 1038, 1048, 1058, 1068, 1078, 1088, 1098, 1108, 1118, 1128, 1138, 1148, 1158, 1168, 1178, 1188, 1198, 1208, 1218, 1228, 1238, 1248, 1258, 1268, 1278, 1288, 1298, 1308, 1318, 1328, 1338, 1348, 1358, 1368, 1378, 1388, 1398, 1408, 1418, 1428, 1438, 1448, 1458, 1468, 1478, 1488, 1498, 1508, 1518, 1528, 1538, 1548, 1558, 1568, 1578, 1588, 1598, 1608, 1618, 1628, 1638, 1648, 1658, 1668, 1678, 1688, 1698, 1708, 1718, 1728, 1738, 1748, 1758, 1768, 1778, 1788, 1798, 1808, 1818, 1828, 1838, 1848, 1858, 1868, 1878, 1888, 1898, 1908, 2163, 2168, 2173, 2178, 2183, 2188, 2193, 2198, 2203, 2208, 2213, 2218, 2223, 2228, 2233, 2238, 2243, 2248, 2253, 2258, 2263, 2268, 2273, 2278, 2283, 2288, 2293, 2298, 2303, 2308, 2313, 2318, 2323, 2328, 2333, or 2338.

In one aspect, the antibody comprises: a heavy chain comprising the amino acid sequence of any one of the following SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714, 724, 734, 744, 754, 764, 774, 784, 794, 804, 814, 824, 834, 844, 854, 864, 874, 884, 894, 904, 914, 924, 934, 944, 954, 964, 974, 984, 994, 1004, 1014, 1024, 1034, 1044, 1054, 1064, 1074, 1084, 1094, 1104, 1114, 1124, 1134, 1144, 1154, 1164, 1174, 1184, 1194, 1204, 1214, 1224, 1234, 1244, 1254, 1264, 1274, 1284, 1294, 1304, 1314, 1324, 1334, 1344, 1354, 1364, 1374, 1384, 1394, 1404, 1414, 1424, 1434, 1444, 1454, 1464, 1474, 1484, 1494, 1504, 1514, 1524, 1534, 1544, 1554, 1564, 1574, 1584, 1594, 1604, 1614, 1624, 1634, 1644, 1654, 1664, 1674, 1684, 1694, 1704, 1714, 1724, 1734, 1744, 1754, 1764, 1774, 1784, 1794, 1804, 1814, 1824, 1834, 1844, 1854, 1864, 1874, 1884, 1894, 1904, 1914, 1919, 1924, 1929, 1934, 1939, 1944, 1949, 1954, 1959, 1964, 1969, 1974, 1979, 1984, 1989, 1994, 1999, 2004, 2009, 2014, 2019, 2024, 2029, 2034, 2039, 2044, 2049, 2054, 2059, 2064, 2069, 2074, 2079, 2084, 2089, 2094, 2099, 2104, 2109, 2114, 2119, 2124, 2129, 2134, 2139, 2144, 2149, 2154, 2159, 2164, 2169, 2174, 2179, 2184, or 2189, and a light chain comprising the amino acid sequence of any one of the following SEQ ID NOs: 9, 19, 29, 39, 49, 59, 69, 79, 89, 99, 109, 119, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 509, 519, 529, 539, 549, 559, 569, 579, 589, 599, 609, 619, 629, 639, 649, 659, 669, 679, 689, 699, 709, 719, 729, 739, 749, 759, 769, 779, 789, 799, 809, 819, 829, 839, 849, 859, 869, 879, 889, 899, 909, 919, 929, 939, 949, 959, 969, 979, 989, 999, 1009, 1019, 1029, 1039, 1049, 1059, 1069, 1079, 1089, 1099, 1109, 1119, 1129, 1139, 1149, 1159, 1169, 1179, 1189, 1199, 1209, 1219, 1229, 1239, 1249, 1259, 1269, 1279, 1289, 1299, 1309, 1319, 1329, 1339, 1349, 1359, 1369, 1379, 1389, 1399, 1409, 1419, 1429, 1439, 1449, 1459, 1469, 1479, 1489, 1499, 1509, 1519, 1529, 1539, 1549, 1559, 1569, 1579, 1589, 1599, 1609, 1619, 1629, 1639, 1649, 1659, 1669, 1679, 1689, 1699, 1709, 1719, 1729, 1739, 1749, 1759, 1769, 1779, 1789, 1799, 1809, 1819, 1829, 1839, 1849, 1859, 1869, 1879, 1889, 1899, 1909, 2164, 2169, 2174, 2179, 2184, 2189, 2194, 2199, 2204, 2209, 2214, 2219, 2224, 2229, 2234, 2239, 2244, 2249, 2254, 2259, 2264, 2269, 2274, 2279, 2284, 2289, 2294, 2299, 2304, 2309, 2314, 2319, 2324, 2329, 2334, or 2339. In another aspect, the antibody is encoded by a nucleic acid that comprises: a VH comprising the nucleic acid sequence of any one of the following SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, 805, 815, 825, 835, 845, 855, 865, 875, 885, 895, 905, 915, 925, 935, 945, 955, 965, 975, 985, 995, 1005, 1015, 1025, 1035, 1045, 1055, 1065, 1075, 1085, 1095, 1105, 1115, 1125, 1135, 1145, 1155, 1165, 1175, 1185, 1195, 1205, 1215, 1225, 1235, 1245, 1255, 1265, 1275, 1285, 1295, 1305, 1315, 1325, 1335, 1345, 1355, 1365, 1375, 1385, 1395, 1405, 1415, 1425, 1435, 1445, 1455, 1465, 1475, 1485, 1495, 1505, 1515, 1525, 1535, 1545, 1555, 1565, 1575, 1585, 1595, 1605, 1615, 1625, 1635, 1645, 1655, 1665, 1675, 1685, 1695, 1705, 1715, 1725, 1735, 1745, 1755, 1765, 1775, 1785, 1795, 1805, 1815, 1825, 1835, 1845, 1855, 1865, 1875, 1885, 1895, 1905, 1915, 1920, 1925, 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, or 2190, and a VL comprising the nucleic acid sequence of any one of the following SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, or 2340. In another aspect, the antibody comprises: a heavy chain variable domain (VH) comprising complementarity determining regions CDR1 to CDR3, comprising the amino acid sequence triplets: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; 491, 492, 493; 501, 502, 503; 511, 512, 513; 521, 522, 523; 531, 532, 533; 541, 542, 543; 551, 552, 553; 561, 562, 563; 571, 572, 573; 581, 582, 583; 591, 592, 593; 601, 602, 603; 611, 612, 613; 621, 622, 623; 631, 632, 633; 641, 642, 643; 651, 652, 653; 661, 662, 663; 671, 672, 673; 681, 682, 683; 691, 692, 693; 701, 702, 703; 711, 712, 713; 721, 722, 723; 731, 732, 733; 741, 742, 743; 751, 752, 753; 761, 762, 763; 771, 772, 773; 781, 782, 783; 791, 792, 793; 801, 802, 803; 811, 812, 813; 821, 822, 823; 831, 832, 833; 841, 842, 843; 851, 852, 853; 861, 862, 863; 871, 872, 873; 881, 882, 883; 891, 892, 893; 901, 902, 903; 911, 912, 913; 921, 922, 923; 931, 932, 933; 941, 942, 943; 951, 952, 953; 961, 962, 963; 971, 972, 973; 981, 982, 983; 991, 992, 993; 1001, 1002, 1003; 1011, 1012, 1013; 1021, 1022, 1023; 1031, 1032, 1033; 1041, 1042, 1043; 1051, 1052, 1053; 1061, 1062, 1063; 1071, 1072, 1073; 1081, 1082, 1083; 1091, 1092, 1093; 1101, 1102, 1103; 1111, 1112, 1113; 1121, 1122, 1123; 1131, 1132, 1133; 1141, 1142, 1143; 1151, 1152, 1153; 1161, 1162, 1163; 1171, 1172, 1173; 1181, 1182, 1183; 1191, 1192, 1193; 1201, 1202, 1203; 1211, 1212, 1213; 1221, 1222, 1223; 1231, 1232, 1233; 1241, 1242, 1243; 1251, 1252, 1253; 1261, 1262, 1263; 1271, 1272, 1273; 1281, 1282, 1283; 1291, 1292, 1293; 1301, 1302, 1303; 1311, 1312, 1313; 1321, 1322, 1323; 1331, 1332, 1333; 1341, 1342, 1343; 1351, 1352, 1353; 1361, 1362, 1363; 1371, 1372, 1373; 1381, 1382, 1383; 1391, 1392, 1393; 1401, 1402, 1403; 1411, 1412, 1413; 1421, 1422, 1423; 1431, 1432, 1433; 1441, 1442, 1443; 1451, 1452, 1453; 1461, 1462, 1463; 1471, 1472, 1473; 1481, 1482, 1483; 1491, 1492, 1493; 1501, 1502, 1503; 1511, 1512, 1513; 1521, 1522, 1523; 1531, 1532, 1533; 1541, 1542, 1543; 1551, 1552, 1553; 1561, 1562, 1563; 1571, 1572, 1573; 1581, 1582, 1583; 1591, 1592, 1593; 1601, 1602, 1603; 1611, 1612, 1613; 1621, 1622, 1623; 1631, 1632, 1633; 1641, 1642, 1643; 1651, 1652, 1653; 1661, 1662, 1663; 1671, 1672, 1673; 1681, 1682, 1683; 1691, 1692, 1693; 1701, 1702, 1703; 1711, 1712, 1713; 1721, 1722, 1723; 1731, 1732, 1733; 1741, 1742, 1743; 1751, 1752, 1753; 1761, 1762, 1763; 1771, 1772, 1773; 1781, 1782, 1783; 1791, 1792, 1793; 1801, 1802, 1803; 1811, 1812, 1813; 1821, 1822, 1823; 1831, 1832, 1833; 1841, 1842, 1843; 1851, 1852, 1853; 1861, 1862, 1863; 1871, 1872, 1873; 1881, 1882, 1883; 1891, 1892, 1893; 1901, 1902, 1903; 1911, 1912, 1913; 1921, 1922, 1923; or 1931, 1932, 1933; and a light chain variable domain (VL) comprising complementarity determining regions CDR1 to CDR3, comprising the amino acid sequence triplets: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; 256, 257, 258; 266, 267, 268; 276, 277, 278; 286, 287, 288; 296, 297, 298; 306, 307, 308; 316, 317, 318; 326, 327, 328; 336, 337, 338; 346, 347, 348; 356, 357, 358; 366, 367, 368; 376, 377, 378; 386, 387, 388; 396, 397, 398; 406, 407, 408; 416, 417, 418; 426, 427, 428; 436, 437, 438; 446, 447, 448; 456, 457, 458; 466, 467, 468; 476, 477, 478; 486, 487, 488; 496, 497, 498; 506, 507, 508; 516, 517, 518; 526, 527, 528; 536, 537, 538; 546, 547, 548; 556, 557, 558; 566, 567, 568; 576, 577, 578; 586, 587, 588; 596, 597, 598; 606, 607, 608; 616, 617, 618; 626, 627, 628; 636, 637, 638; 646, 647, 648; 656, 657, 658; 666, 667, 668; 676, 677, 678; 686, 687, 688; 696, 697, 698; 706, 707, 708; 716, 717, 718; 726, 727, 728; 736, 737, 738; 746, 747, 748; 756, 757, 758; 766, 767, 768; 776, 777, 778; 786, 787, 788; 796, 797, 798; 806, 807, 808; 816, 817, 818; 826, 827, 828; 836, 837, 838; 846, 847, 848; 856, 857, 858; 866, 867, 868; 876, 877, 878; 886, 887, 888; 896, 897, 898; 906, 907, 908; 916, 917, 918; 926, 927, 928; 936, 937, 938; 946, 947, 948; 956, 957, 958; 966, 967, 968; 976, 977, 978; 986, 987, 988; 996, 997, 998; 1006, 1007, 1008; 1016, 1017, 1018; 1026, 1027, 1028; 1036, 1037, 1038; 1046, 1047, 1048; 1056, 1057, 1058; 1066, 1067, 1068; 1076, 1077, 1078; 1086, 1087, 1088; 1096, 1097, 1098; 1106, 1107, 1108; 1116, 1117, 1118; 1126, 1127, 1128; 1136, 1137, 1138; 1146, 1147, 1148; 1156, 1157, 1158; 1166, 1167, 1168; 1176, 1177, 1178; 1186, 1187, 1188; 1196, 1197, 1198; 1206, 1207, 1208; 1216, 1217, 1218; 1226, 1227, 1228; 1236, 1237, 1238; 1246, 1247, 1248; 1256, 1257, 1258; 1266, 1267, 1268; 1276, 1277, 1278; 1286, 1287, 1288; 1296, 1297, 1298; 1306, 1307, 1308; 1316, 1317, 1318; 1326, 1327, 1328; 1336, 1337, 1338; 1346, 1347, 1348; 1356, 1357, 1358; 1366, 1367, 1368; 1376, 1377, 1378; 1386, 1387, 1388; 1396, 1397, 1398; 1406, 1407, 1408; 1416, 1417, 1418; 1426, 1427, 1428; 1436, 1437, 1438; 1446, 1447, 1448; 1456, 1457, 1458; 1466, 1467, 1468; 1476, 1477, 1478; 1486, 1487, 1488; 1496, 1497, 1498; 1506, 1507, 1508; 1516, 1517, 1518; 1526, 1527, 1528; 1536, 1537, 1538; 1546, 1547, 1548; 1556, 1557, 1558; 1566, 1567, 1568; 1576, 1577, 1578; 1586, 1587, 1588; 1596, 1597, 1598; 1606, 1607, 1608; 1616, 1617, 1618; 1626, 1627, 1628; 1636, 1637, 1638; 1646, 1647, 1648; 1656, 1657, 1658; 1666, 1667, 1668; 1676, 1677, 1678; 1686, 1687, 1688; 1696, 1697, 1698; 1706, 1707, 1708; 1716, 1717, 1718; 1726, 1727, 1728; 1736, 1737, 1738; 1746, 1747, 1748; 1756, 1757, 1758; 1766, 1767, 1768; 1776, 1777, 1778; 1786, 1787, 1788; 1796, 1797, 1798; 1806, 1807, 1808; 1816, 1817, 1818; 1826, 1827, 1828; 1836, 1837, 1838; 1846, 1847, 1848; 1856, 1857, 1858; 1866, 1867, 1868; 1876, 1877, 1878; 1886, 1887, 1888; 1896, 1897, 1898; 1906, 1907, 1908; 1916, 1917, 1918; 1926, 1927, 1928; or 1936, 1937, 1938. In another aspect, the antibody comprises: a heavy chain variable domain (VH) comprising complementarity determining regions CDR1 to CDR3, comprising the amino acid sequence triplets: 1941, 1942, 1943; 1946, 1947, 1948; 1951, 1952, 1953; 1956, 1957, 1958; 1961, 1962, 1963; 1966, 1967, 1968; 1971, 1972, 1973; 1976, 1977, 1978; 1981, 1982, 1983; 1986, 1987, 1988; 1991, 1992, 1993; 1996, 1997, 1998; 2001, 2002, 2003; 2006, 2007, 2008; 2011, 2012, 2013; 2016, 2017, 2018; 2021, 2022, 2023; 2026, 2027, 2028; 2031, 2032, 2033; 2036, 2037, 2038; 2041, 2042, 2043; 2046, 2047, 2048; 2051, 2052, 2053; 2056, 2057, 2058; 2061, 2062, 2063; 2066, 2067, 2068; 2071, 2072, 2073; 2076, 2077, 2078; 2081, 2082, 2083; 2086, 2087, 2088; 2091, 2092, 2093; 2096, 2097, 2098; 2101, 2102, 2103; 2106, 2107, 2108; 2111, 2112, 2113; 2116, 2117, 2118; 2121, 2122, 2123; 2126, 2127, 2128; 2131, 2132, 2133; 2136, 2137, 2138; 2141, 2142, 2143; 2146, 2147, 2148; 2151, 2152, 2153; 2156, 2157, 2158; 2161, 2162, 2163; 2166, 2167, 2168; 2171, 2172, 2173; 2176, 2177, 2178; 2181, 2182, 2183; or 2186, 2187, 2188; and a light chain variable domain (VL) comprising complementarity determining regions CDR1 to CDR3, comprising the amino acid sequence triplets: 2191, 2192, 2193; 2196, 2197, 2198; 2201, 2202, 2203; 2206, 2207, 2208; 2211, 2212, 2213; 2216, 2217, 2218; 2221, 2222, 2223; 2226, 2227, 2228; 2231, 2232, 2233; 2236, 2237, 2238; 2241, 2242, 2243; 2246, 2247, 2248; 2251, 2252, 2253; 2256, 2257, 2258; 2261, 2262, 2263; 2266, 2267, 2268; 2271, 2272, 2273; 2276, 2277, 2278; 2281, 2282, 2283; 2286, 2287, 2288; 2291, 2292, 2293; 2296, 2297, 2298; 2301, 2302, 2303; 2306, 2307, 2308; 2311, 2312, 2313; 2316, 2317, 2318; 2321, 2322, 2323; 2326, 2327, 2328; 2331, 2332, 2333; or 2336, 2337, 2338. In another aspect, the antibody is a monoclonal, bispecific, multivalent, multi-specific, diabody, chimeric, scFv antibody, or fragments thereof. In another aspect, the antibody is a full-length antibody. In another aspect, the antibody is an antibody fragment. In another aspect, the antibody is fused to an Fc domain of any one of the following: human IgG1, human IgG2, human IgG3, and human IgG4. In another aspect, the Fc domain is a wild-type, variant, or truncated Fc domain. In another aspect, the antibody further comprises a peptide linker between a VH2 domain and the CH1 domain.

As embodied and broadly described herein, an aspect of the present disclosure relates to a method of treating a disease in a subject in need thereof, comprising administering to the subject a therapeutically effective amount of the antibodies, binding fragments, bi- or multi-valent antibodies, chimeric proteins, scFv, etc. described hereinabove. In one aspect, the disease is an autoimmune disease, an inflammatory disease, or a cancer. In another aspect, the subject is human.

As embodied and broadly described herein, an aspect of the present disclosure relates to a nucleic acid encoding an anti-CD3 antibody, wherein the nucleic acid comprises: a VH comprising the nucleic acid sequence of any one of the following SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, 805, 815, 825, 835, 845, 855, 865, 875, 885, 895, 905, 915, 925, 935, 945, 955, 965, 975, 985, 995, 1005, 1015, 1025, 1035, 1045, 1055, 1065, 1075, 1085, 1095, 1105, 1115, 1125, 1135, 1145, 1155, 1165, 1175, 1185, 1195, 1205, 1215, 1225, 1235, 1245, 1255, 1265, 1275, 1285, 1295, 1305, 1315, 1325, 1335, 1345, 1355, 1365, 1375, 1385, 1395, 1405, 1415, 1425, 1435, 1445, 1455, 1465, 1475, 1485, 1495, 1505, 1515, 1525, 1535, 1545, 1555, 1565, 1575, 1585, 1595, 1605, 1615, 1625, 1635, 1645, 1655, 1665, 1675, 1685, 1695, 1705, 1715, 1725, 1735, 1745, 1755, 1765, 1775, 1785, 1795, 1805, 1815, 1825, 1835, 1845, 1855, 1865, 1875, 1885, 1895, 1905, 1915, 1920, 1925, 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, or 2190, and a VL comprising the nucleic acid sequence of any one of the following SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, or 2340.

As embodied and broadly described herein, an aspect of the present disclosure relates to an expression vector comprising a nucleic acid encoding at least one of a nucleic acids encoding the antibodies, binding fragments, bi- or multi-valent antibodies, chimeric proteins, scFv, etc. described hereinabove. Also included are one or more host cells comprising the vector(s) described hereinabove.

As embodied and broadly described herein, an aspect of the present disclosure relates to a method of making an anti-CD3 antibody comprising: expressing in a cell a heavy chain variable domain (VH) comprising complementarity determining regions CDR1 to CDR3, comprising the amino acid sequence triplets: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; 491, 492, 493; 501, 502, 503; 511, 512, 513; 521, 522, 523; 531, 532, 533; 541, 542, 543; 551, 552, 553; 561, 562, 563; 571, 572, 573; 581, 582, 583; 591, 592, 593; 601, 602, 603; 611, 612, 613; 621, 622, 623; 631, 632, 633; 641, 642, 643; 651, 652, 653; 661, 662, 663; 671, 672, 673; 681, 682, 683; 691, 692, 693; 701, 702, 703; 711, 712, 713; 721, 722, 723; 731, 732, 733; 741, 742, 743; 751, 752, 753; 761, 762, 763; 771, 772, 773; 781, 782, 783; 791, 792, 793; 801, 802, 803; 811, 812, 813; 821, 822, 823; 831, 832, 833; 841, 842, 843; 851, 852, 853; 861, 862, 863; 871, 872, 873; 881, 882, 883; 891, 892, 893; 901, 902, 903; 911, 912, 913; 921, 922, 923; 931, 932, 933; 941, 942, 943; 951, 952, 953; 961, 962, 963; 971, 972, 973; 981, 982, 983; 991, 992, 993; 1001, 1002, 1003; 1011, 1012, 1013; 1021, 1022, 1023; 1031, 1032, 1033; 1041, 1042, 1043; 1051, 1052, 1053; 1061, 1062, 1063; 1071, 1072, 1073; 1081, 1082, 1083; 1091, 1092, 1093; 1101, 1102, 1103; 1111, 1112, 1113; 1121, 1122, 1123; 1131, 1132, 1133; 1141, 1142, 1143; 1151, 1152, 1153; 1161, 1162, 1163; 1171, 1172, 1173; 1181, 1182, 1183; 1191, 1192, 1193; 1201, 1202, 1203; 1211, 1212, 1213; 1221, 1222, 1223; 1231, 1232, 1233; 1241, 1242, 1243; 1251, 1252, 1253; 1261, 1262, 1263; 1271, 1272, 1273; 1281, 1282, 1283; 1291, 1292, 1293; 1301, 1302, 1303; 1311, 1312, 1313; 1321, 1322, 1323; 1331, 1332, 1333; 1341, 1342, 1343; 1351, 1352, 1353; 1361, 1362, 1363; 1371, 1372, 1373; 1381, 1382, 1383; 1391, 1392, 1393; 1401, 1402, 1403; 1411, 1412, 1413; 1421, 1422, 1423; 1431, 1432, 1433; 1441, 1442, 1443; 1451, 1452, 1453; 1461, 1462, 1463; 1471, 1472, 1473; 1481, 1482, 1483; 1491, 1492, 1493; 1501, 1502, 1503; 1511, 1512, 1513; 1521, 1522, 1523; 1531, 1532, 1533; 1541, 1542, 1543; 1551, 1552, 1553; 1561, 1562, 1563; 1571, 1572, 1573; 1581, 1582, 1583; 1591, 1592, 1593; 1601, 1602, 1603; 1611, 1612, 1613; 1621, 1622, 1623; 1631, 1632, 1633; 1641, 1642, 1643; 1651, 1652, 1653; 1661, 1662, 1663; 1671, 1672, 1673; 1681, 1682, 1683; 1691, 1692, 1693; 1701, 1702, 1703; 1711, 1712, 1713; 1721, 1722, 1723; 1731, 1732, 1733; 1741, 1742, 1743; 1751, 1752, 1753; 1761, 1762, 1763; 1771, 1772, 1773; 1781, 1782, 1783; 1791, 1792, 1793; 1801, 1802, 1803; 1811, 1812, 1813; 1821, 1822, 1823; 1831, 1832, 1833; 1841, 1842, 1843; 1851, 1852, 1853; 1861, 1862, 1863; 1871, 1872, 1873; 1881, 1882, 1883; 1891, 1892, 1893; 1901, 1902, 1903; 1911, 1912, 1913; 1921, 1922, 1923; or 1931, 1932, 1933; and a light chain variable domain (VL) comprising complementarity determining regions CDR1 to CDR3, comprising the amino acid sequence triplets: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; 256, 257, 258; 266, 267, 268; 276, 277, 278; 286, 287, 288; 296, 297, 298; 306, 307, 308; 316, 317, 318; 326, 327, 328; 336, 337, 338; 346, 347, 348; 356, 357, 358; 366, 367, 368; 376, 377, 378; 386, 387, 388; 396, 397, 398; 406, 407, 408; 416, 417, 418; 426, 427, 428; 436, 437, 438; 446, 447, 448; 456, 457, 458; 466, 467, 468; 476, 477, 478; 486, 487, 488; 496, 497, 498; 506, 507, 508; 516, 517, 518; 526, 527, 528; 536, 537, 538; 546, 547, 548; 556, 557, 558; 566, 567, 568; 576, 577, 578; 586, 587, 588; 596, 597, 598; 606, 607, 608; 616, 617, 618; 626, 627, 628; 636, 637, 638; 646, 647, 648; 656, 657, 658; 666, 667, 668; 676, 677, 678; 686, 687, 688; 696, 697, 698; 706, 707, 708; 716, 717, 718; 726, 727, 728; 736, 737, 738; 746, 747, 748; 756, 757, 758; 766, 767, 768; 776, 777, 778; 786, 787, 788; 796, 797, 798; 806, 807, 808; 816, 817, 818; 826, 827, 828; 836, 837, 838; 846, 847, 848; 856, 857, 858; 866, 867, 868; 876, 877, 878; 886, 887, 888; 896, 897, 898; 906, 907, 908; 916, 917, 918; 926, 927, 928; 936, 937, 938; 946, 947, 948; 956, 957, 958; 966, 967, 968; 976, 977, 978; 986, 987, 988; 996, 997, 998; 1006, 1007, 1008; 1016, 1017, 1018; 1026, 1027, 1028; 1036, 1037, 1038; 1046, 1047, 1048; 1056, 1057, 1058; 1066, 1067, 1068; 1076, 1077, 1078; 1086, 1087, 1088; 1096, 1097, 1098; 1106, 1107, 1108; 1116, 1117, 1118; 1126, 1127, 1128; 1136, 1137, 1138; 1146, 1147, 1148; 1156, 1157, 1158; 1166, 1167, 1168; 1176, 1177, 1178; 1186, 1187, 1188; 1196, 1197, 1198; 1206, 1207, 1208; 1216, 1217, 1218; 1226, 1227, 1228; 1236, 1237, 1238; 1246, 1247, 1248; 1256, 1257, 1258; 1266, 1267, 1268; 1276, 1277, 1278; 1286, 1287, 1288; 1296, 1297, 1298; 1306, 1307, 1308; 1316, 1317, 1318; 1326, 1327, 1328; 1336, 1337, 1338; 1346, 1347, 1348; 1356, 1357, 1358; 1366, 1367, 1368; 1376, 1377, 1378; 1386, 1387, 1388; 1396, 1397, 1398; 1406, 1407, 1408; 1416, 1417, 1418; 1426, 1427, 1428; 1436, 1437, 1438; 1446, 1447, 1448; 1456, 1457, 1458; 1466, 1467, 1468; 1476, 1477, 1478; 1486, 1487, 1488; 1496, 1497, 1498; 1506, 1507, 1508; 1516, 1517, 1518; 1526, 1527, 1528; 1536, 1537, 1538; 1546, 1547, 1548; 1556, 1557, 1558; 1566, 1567, 1568; 1576, 1577, 1578; 1586, 1587, 1588; 1596, 1597, 1598; 1606, 1607, 1608; 1616, 1617, 1618; 1626, 1627, 1628; 1636, 1637, 1638; 1646, 1647, 1648; 1656, 1657, 1658; 1666, 1667, 1668; 1676, 1677, 1678; 1686, 1687, 1688; 1696, 1697, 1698; 1706, 1707, 1708; 1716, 1717, 1718; 1726, 1727, 1728; 1736, 1737, 1738; 1746, 1747, 1748; 1756, 1757, 1758; 1766, 1767, 1768; 1776, 1777, 1778; 1786, 1787, 1788; 1796, 1797, 1798; 1806, 1807, 1808; 1816, 1817, 1818; 1826, 1827, 1828; 1836, 1837, 1838; 1846, 1847, 1848; 1856, 1857, 1858; 1866, 1867, 1868; 1876, 1877, 1878; 1886, 1887, 1888; 1896, 1897, 1898; 1906, 1907, 1908; 1916, 1917, 1918; 1926, 1927, 1928; or 1936, 1937, 1938; or a heavy chain variable domain (VH) comprising complementarity determining regions CDR1 to CDR3, comprising the amino acid sequence triplets: 1941, 1942, 1943; 1946, 1947, 1948; 1951, 1952, 1953; 1956, 1957, 1958; 1961, 1962, 1963; 1966, 1967, 1968; 1971, 1972, 1973; 1976, 1977, 1978; 1981, 1982, 1983; 1986, 1987, 1988; 1991, 1992, 1993; 1996, 1997, 1998; 2001, 2002, 2003; 2006, 2007, 2008; 2011, 2012, 2013; 2016, 2017, 2018; 2021, 2022, 2023; 2026, 2027, 2028; 2031, 2032, 2033; 2036, 2037, 2038; 2041, 2042, 2043; 2046, 2047, 2048; 2051, 2052, 2053; 2056, 2057, 2058; 2061, 2062, 2063; 2066, 2067, 2068; 2071, 2072, 2073; 2076, 2077, 2078; 2081, 2082, 2083; 2086, 2087, 2088; 2091, 2092, 2093; 2096, 2097, 2098; 2101, 2102, 2103; 2106, 2107, 2108; 2111, 2112, 2113; 2116, 2117, 2118; 2121, 2122, 2123; 2126, 2127, 2128; 2131, 2132, 2133; 2136, 2137, 2138; 2141, 2142, 2143; 2146, 2147, 2148; 2151, 2152, 2153; 2156, 2157, 2158; 2161, 2162, 2163; 2166, 2167, 2168; 2171, 2172, 2173; 2176, 2177, 2178; 2181, 2182, 2183; or 2186, 2187, 2188; and a light chain variable domain (VL) comprising complementarity determining regions CDR1 to CDR3, comprising the amino acid sequence triplets: 2191, 2192, 2193; 2196, 2197, 2198; 2201, 2202, 2203; 2206, 2207, 2208; 2211, 2212, 2213; 2216, 2217, 2218; 2221, 2222, 2223; 2226, 2227, 2228; 2231, 2232, 2233; 2236, 2237, 2238; 2241, 2242, 2243; 2246, 2247, 2248; 2251, 2252, 2253; 2256, 2257, 2258; 2261, 2262, 2263; 2266, 2267, 2268; 2271, 2272, 2273; 2276, 2277, 2278; 2281, 2282, 2283; 2286, 2287, 2288; 2291, 2292, 2293; 2296, 2297, 2298; 2301, 2302, 2303; 2306, 2307, 2308; 2311, 2312, 2313; 2316, 2317, 2318; 2321, 2322, 2323; 2326, 2327, 2328; 2331, 2332, 2333; or 2336, 2337, 2338. In one aspect, the antibody is a monoclonal, bispecific, multivalent, multi-specific, diabody, chimeric, scFv antibody, or fragments thereof. In another aspect, the antibody is a full-length antibody. In another aspect, the antibody is an antibody fragment. In another aspect, the e antibody is fused to an Fc domain of any one of the following: human IgG1, human IgG2, human IgG3, and human IgG4. In another aspect, the Fc domain is a wild-type, variant, or truncated Fc domain. In another aspect, the antibodies, binding fragments, bi- or multi-valent antibodies, chimeric proteins, scFv, etc. described hereinabove further comprise a peptide linker between a VH2 domain and the CH1 domain. For each of the above amino acid and nucleic acid sequences, the present invention includes those that have at least 91, 92, 93, 94, 95, 96, 97, 98, 99 percent identify at the amino acid or nucleic acid level, and nucleic acid sequences optimized for expression in different host cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present application can be understood by reference to the following description taking in conjunction with the accompanying figures.

FIGS. 1A and 1B illustrates the two paths to anti-CD3 specific antibody discovery (FIG. 1A) and the method of screening (FIG. 1B).

FIG. 2 shows the engineered epitopes steered immunization to three CD3E & CD3D epitopes.

FIG. 3 shows the immunized cohorts CD3 hybridoma primary screens.

FIG. 4 is a graph that shows epitope-steered immunization and StableHu generated CD3 hits across a broad binding range.

FIG. 5 shows that the StableHu generated a panel of CD3 Hits with a range of binding affinities.

FIG. 6 shows that the immunized CD3 repertoire was cloned into mammalian display for demultiplexed screening.

FIG. 7 shows the results from the first round of mammalian display screening of multi-epitope immunized CD3 repertoire.

FIG. 8 shows the results from the second round of immunized CD3 mammalian display repertoire that is significantly enriched to binders.

FIG. 9 shows that the ELISA screening identifies Hu-Cyno cross-reactive T-Cell binders.

FIG. 10 shows that StableHu generates a library of Hu-Cyno cross-reactivity from anti-CD3 murine CDR template. Significant overlap in Hu-Cyno library binding distributions, ˜50-fold binding range for 86 fully human CDR hits in-testing.

FIG. 11 shows cell sorting identifies range of Hu-Cyno Cross Reactive CD3 binders, with sequences rank-ordered from bin-weight analysis.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

As used herein, the term “antibody” refers to an intact antibody or a binding fragment thereof that binds specifically to a target antigen. Binding fragments are produced by recombinant DNA techniques, or by enzymatic or chemical cleavage of intact antibodies. Binding fragments include Fab, Fab′, F(ab′)2, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)2, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen-binding or variable region, and include Fab, Fab′, F(ab′)2, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)2 fragment that has two antigen-binding fragments which are capable of cross-linking antigen, and a residual other fragment (which is termed pFc′). As used herein, “functional fragment” with respect to antibodies, refers to Fv, F(ab) and F(ab′)2 fragments. As used herein, the “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

In some embodiments, bispecific or multispecific antibodies are provided, which comprise at least a heavy chain variable region from the antibody family of the invention and may comprise a heavy and light chain variable region provided herein. Bispecific antibodies comprise at least the heavy chain variable region of an antibody specific for a protein other than CD3, and may comprise a heavy and light chain variable region. In some such embodiments, the second antibody specificity binds to a tumor associated antigen, a targeting antigen, e.g., integrins, etc., a pathogen antigen, a checkpoint protein, and the like. Various formats of bispecific antibodies are within the scope of the invention, including without limitation single chain polypeptides, two chain polypeptides, three chain polypeptides, four chain polypeptides, and multiples thereof.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (VH) followed by the constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342:877), or both, that is Chothia plus Kabat. The more highly conserved portions of variable domains are called the framework (FR). The variable domains of native heavy and light chains each comprise four FR regions, largely adopting a ß-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

As used herein, the term “binding affinity” refers to the strength of the sum total of noncovalent interactions between a single binding site of a molecule (e.g., an antibody or other binding molecule) and its binding partner (e.g., an antigen or receptor). The affinity of a molecule X for its partner Y can generally be represented by the dissociation constant (Kd). Affinity can be measured by common methods known in the art, including those described herein. Low-affinity antibodies bind antigen (or receptor) weakly and tend to dissociate readily, whereas high-affinity antibodies bind antigen (or receptor) more tightly and remain bound longer. In the case of antibodies, the antigen binding site binds to its cognate epitope.

As used herein, the terms “cancer,” “neoplasm,” and “tumor” are used interchangeably to refer to cells that exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. As used herein, the phrase “cancer burden” refers to the number of cancer cells or cancer volume in a subject. Reducing a cancer burden refers to reducing the number of cancer cells or the cancer volume in a subject. As used herein, the term “cancer cell” refers to any cell that is a cancer cell or is derived from a cancer cell, e.g., a clone of a cancer cell. Many types of cancers are known to those of skill in the art, including solid tumors such as carcinomas, sarcomas, glioblastomas, melanomas, lymphomas, myelomas, etc., and circulating cancers such as leukemias, including specifically B cell leukemias, T cell leukemias, etc. Examples of cancer that may be treated with the anti-CD3 antibodies include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for the CD3 is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)2, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

As used herein, the term “chimeric” antibody refers to a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. A chimeric antibody will have two distinct antigen binding sites, which may be against the same or different antigens.

Antibody fragments of the disclosure retain anti-CD3 antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

The term “meso scale-molecule (MEM)” as used herein throughout includes engineered peptides and polypeptides between about 1 kDa and about 10 kDa. The term “MEM-nanoparticle” as used herein throughout includes MEMs which have been conjugated to a nanoparticle (e.g., ferritin nanoparticle).

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

The presently disclosed and claimed invention further includes the use of fully human monoclonal antibodies cross-reactive against CD3. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “vector” refers to a nucleic acid molecule capable of transporting another nucleic acid to which it has been linked. One type of vector is a “plasmid”, which refers to a circular double-stranded DNA into which additional DNA segments are inserted. Another type of vector is a viral vector in which DNA segments are inserted into a viral genome. Certain vectors are capable of autonomous replication in a host cell into which they are introduced (e.g., bacterial vectors having a bacterial and an episomal origin of replication, or non-episomal mammalian vectors can be integrated into the genome of a host cell upon introduction into the host cell, and thereby are replicated along with the host genome. Certain vectors are capable of directing the expression of genes to which they are operably linked. Such vectors are referred to herein as “recombinant expression vectors” (or simply, “recombinant vectors”). Generally, expression vectors useful for expression in mammalian cells using recombinant DNA techniques are often in the form of plasmids.

As used herein, the term “host cell” or “recombinant host cell” refer to a cell that has been genetically altered, or is capable of being genetically altered by introduction of an exogenous polynucleotide, such as a recombinant plasmid or vector. A host cell refers not only to a particular subject cell but also to the progeny of such a host cell. Because certain modifications may occur in succeeding generations due to either mutation or environmental influences, such progeny may not, in fact, be identical to the parent cell, but are still included within the scope of the term host cell.

As used herein, the term “treatment” refers to both therapeutic treatment and prophylactic or preventative measures. Those in need of treatment include those already with the disease or disorder as well as those in which the disorder is to be prevented. The effect may be prophylactic in terms of completely or partially preventing a disease or symptom thereof and/or may be therapeutic in terms of a partial or complete cure for a disease and/or adverse effects attributable to the disease. A “treatment” refers to any treatment of a disease or disorder in a mammal and includes: (a) preventing the disease from occurring in a subject that may be predisposed to the disease but has not yet been diagnosed as having it; (b) relieving the disease, i.e., causing regression of the disease; or (c) inhibiting the disease, i.e., arresting its development. The therapeutic agent may be administered before, during, or after the onset of disease or injury. The treatment of ongoing disease, where the treatment stabilizes or reduces the undesirable clinical symptoms of the patient, is of particular interest. Such treatment is desirably performed prior to the complete loss of function in the affected tissues. The subject therapy may be administered during the symptomatic stage of the disease, and in some cases after the symptomatic stage of the disease. Treatment may be delivered prophylactically (prior to showing signs of an autoimmune disease or cancer) or therapeutically (following the presence of an autoimmune disease or cancer).

As used herein, the term “disorder” refers to any condition that would benefit from treatment with the anti-CD3 antibody or fragments thereof. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in W0/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

The Fc region can be a wild-type Fc region, a mutated Fc region, a monomeric wild-type Fc region, a monomeric mutant Fc region, a dimeric wild-type Fc region, or a dimeric mutant Fc region, a second variable heavy region and a second Fc region, or a second variable heavy region and a second Fc region, and may further include an uncleavable flexible linker and a second variable light region and a second heavy variable region, or a second Fc region and an uncleavable flexible linker and payload, such as a toxin, a cytokine, an anti-cancer agent, or another antibody.

Non-limiting examples of payload include, e.g., cytotoxic proteins or peptides include a bacterial cytotoxin such as an alpha-pore forming toxin (e.g., cytolysin A from E. coli), a beta-pore-forming toxin (e.g., α-Hemolysin, PVL-panton Valentine leukocidin, acrolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2).

The payload can also be a cytotoxic agent, such as doxorubicin (adriamycin), gemcitabine (gemzar), daunorubicin, procarbazine, mitomycin, cytarabine, ctoposide, methotrexate, venorelbine, 5-fluorouracil (5-FU), vinca alkaloids such as vinblastine or vincristine; bleomycin, paclitaxel (taxol), docetaxel (taxotere), aldesleukin, asparaginase, busulfan, carboplatin, cladribine, camptothecin, CPT-11, 1O-hydroxy-7-ethylcamptothecin (SN38), dacarbazine, S-I capecitabine, ftorafur, 5′deoxyfluorouridine, UFT, eniluracil, deoxycytidine, 5-azacytosine, 5-azadeoxycytosine, allopurinol, 2-chloroadenosine, trimetrexate, aminopterin, methylene-10-deazaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, and combinations thereof.

Non-limiting examples of cytokines include: growth hormone; parathyroid hormone; thyroxine; insulin; proinsulin; relaxin; prorelaxin; glycoprotein hormones; hepatic growth factor; fibroblast growth factor; prolactin; placental lactogen; TNF-α; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-α; IFN-b; IFN-g; IFN-g inducing factor (IGIF); bone morphogenetic protein (BMP); leukemia inhibitory factor (LIF); or kit ligand (KL).

Non-limiting examples of anti-cancer drug include, e.g., dihydroxyvitamin D3, 4-ipomcanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DLPTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, carn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-NI, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazorurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone Bl, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosafe sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×106 different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (cither linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

The practice of the present invention employs, unless otherwise indicated, conventional methods of chemistry, biochemistry, molecular biology, immunology, and pharmacology, within the skill of the art. Such techniques are explained fully in the literature. Sec, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

The skilled artisan will recognize that antibodies that exhibit little or no binding to a target antigen can be described as having a low affinity, and a high equilibrium dissociation constant (KD) for the target antigen. The skilled artisan will also recognize that antibodies that exhibit little or no binding to a collective assembly of target antigenic epitopes can be described as having a low avidity, and a high equilibrium dissociation constant (KD) for the collective assembly of target antigenic epitopes.

In some embodiments, provided herein are anti-CD3 antibodies having a binding affinity (KD) to CD3 of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about InM to about 5 pM, about 0.5 nM to about 5 pM, about 0.1 nM to about 5 pM, about 50 pM to about 5 pM, about 10 pM to about 5 pM.

In some embodiments, anti-CD3 antibodies have a binding avidity (KD) to CD3 of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, anti-CD3 antibodies have a half maximal effective concentration (EC50) to anti-CD3 of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

In some embodiments, the anti-CD3 antibody is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of an anti-CD3 antibody is from a human IgG1, human IgG2, human IgG3, or human lgG4.

Provided herein are sequences for exemplary anti-CD3 antibodies of the disclosure. Included are complementarity determining region (CDR) sequences and the variable heavy and light domain sequences (VH, VL) that constitute the CD3 antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. The table herein provide exemplary CDR combinations of antibodies of the disclosure.

Each antibody arm may be connected by a linker, e.g., a flexible linker. An exemplary linker comprises the following amino acid sequence: GGGGS (SEQ ID NO:2352), GGGGG (SEQ ID NO:2353), or even GGGGSGGGGSGGGGS (SEQ ID NO:2354).

Uses of anti-CD3 Antibodies. Therapeutic anti-CD3 Antibodies.

In some embodiments, the anti-CD3 antibodies provided herein are useful for the treatment of a disease or condition involving an immune response. In some embodiments, the anti-CD3 antibodies provided herein are useful for the treatment of an autoimmune disease. An autoimmune disease is generally a harmful immune response to a self-antigen. Examples of autoimmune diseases include, e.g., alopecia, ankylosing spondylitis, atopic dermatitis, celiac disease, Crohn's disease, cutaneous lupus erythematosus (CLE), lupus nephritis, multiple sclerosis, neuromyelitis optica, psoriasis, psoriatic arthritis, rheumatoid arthritis, scleroderma, Sjogren's syndrome, systemic lupus, systemic lupus crythematosus (SLE), temporal arteritis, type I diabetes, ulcerative colitis, uveitis, and vitiligo.

In some embodiments, the anti-CD3 antibodies provided herein are useful for the treatment of a hyperinflammatory disease. A hyperinflammatory disease consists of a potentially harmful overstimulated immune response. Examples of hyperinflammatory diseases include, e.g., chronic allergy, hypersensitivity vasculitis, and T cell hypersensitivity disease.

In other embodiments, the anti-CD3 antibodies provided herein are useful for the treatment of cancer. In certain aspect, the anti-CD3 antibodies are used alone or in combination to target the cancer cells. In other example, the anti-CD3 antibodies are used alone or in combination to target regulatory T cells that are reducing or blocking an immune response to the cancer cells.

Administration of Therapeutic anti-CD3 Antibodies.

The in vivo administration of the therapeutic anti-CD3 antibodies described herein may be carried out intravenously, intramuscularly, subcutaneously, topically, orally, transdermally, subdermally, intraperitoneally, intraorbitally, intrathecally, intraventricularly, intranasally, transmucosally, through implantation, or through inhalation. Intravenous administration may be carried out via injection or infusion. In some embodiments, the anti-CD3 antibodies of the disclosure are administered intravenously. In some embodiments, the anti-CD3 antibodies of the disclosure are administered subcutaneously. Administration of the therapeutic anti-CD3 antibodies may be performed with any suitable excipients, carriers, or other agents to provide suitable or improved tolerance, transfer, delivery, and the like.

### EXAMPLES

Engineered Epitope Design. Three engineered epitopes were built to embody solvent-accessible epitopes on the human CD3e and CD3d subunits. The target epitope sequences were PQYPGSEILWQ (SEQ ID NO: 2341) and YPRGSKPEDAN (CD3e residues) (SEQ ID NO: 2342), QDGNEEMGGITQTPYKVSISG (CD3e residues) (SEQ ID NO: 2343), and YRCNGTDIYKDKEST (CD3d residues) (SEQ ID NO: 2344).

Peptide scaffolds were computationally designed to support the native sequence and structure of the human CD3ed epitopes with the iBio Engineered Epitope machine learning engine. The design process starts with an initial set of blueprints indicating the positions of the scaffold residues to design and the positions of the epitope residues to hold fixed. The machine learning engine optimizes the scaffold residues in these initial blueprints for [1] structural match to the native epitope structure, [2] overall structural stability of the molecule, and [3] solubility of the peptide design. The number of scaffold residues in the blueprints are then iteratively removed until the machine learning engine can no longer satisfy all three of the loss functions described above. The final design is based on the blueprint that satisfies all loss functions using the minimal number of scaffold residues. These final sequence designs are listed in Table 1.

TABLE 1

| Engineered | |
|---|---|
| Epitope ID | Sequence |
| 1 | SPQYPGSEILWQKWGNKKVSYPRGSKPEDAN |
| | (SEQ ID NO: 2345) |
| 2 | QDGNEEMGGITQTPYKVSGSG |
| | (SEQ ID NO: 2346) |
| 3 | YRSNGTDIYKDKEST (SEQ ID NO: 2347) |

When the engineered epitopes are made by peptide synthesis a cysteine is introduced into the sequence for conjugation to a maleimide-functionalized carrier protein (keyhole limpet hemocyanin or ferritin) for immunizations. Alternatively, the peptides are biotinylated for antibody screening assays (SPR, ELISA, or T-cell activation assays).

Human CDR Library Designs. Starting with the two benchmark antibodies template Ab1 (Table 2) and template Ab2 (Table 3), the CDR sequences were simultaneously humanized and diversified with the iBio StableHu machine learning engine. The machine learning engine is trained on the Observed Antibody Space (OAS) database with masked language modeling to learn numerical embeddings for the human CDR sequences. These CDR embeddings are then rank ordered by similarity to the benchmark antibody CDR embeddings. This allows for identification of human CDR sequences that may be suitable as substitutes for the benchmark CDRs. The most similar human CDRs identified from the OAS database were used to make CDR libraries with diversities of 1000 or 2000 as shown in Table 4.

TABLE 2

| Chain | Template Ab1 Sequence |
|---|---|
| VH | EVQLVQSGSELKKPGSSVKVSCKASGVTFNSRTFTISWVRQAPGQGLEWL |
| | GSIIPIFGTITYAQKFQGRVTITADKSTSTAFMELTSLRSEDTAIYYCTRRGN |
| | WNPFDPWGQGTLVTVSS (SEQ ID NO: 2348) |
| VL | DIQMTQSPSSLSASVGDRVTITCRASQSISSYLNWYQQKPGKAPKLLIYAA |
| | SSLQSGVPSRFSGSGSGTDFTLTISSLQPEDFATYYCQQSYSTPPTFGQGTK |
| | VEIK (SEQ ID NO: 2349) |

TABLE 3

| Chain | Template Ab2 Sequence |
|---|---|
| VH | EVQLVESGGGLVQPGGSLRLSCAASGFTFNTYAMNWVRQAPGKGLEWV |
| | GRIRSKYNNYATYYADSVKGRFTISRDDSKNTLYLQMNSLRAEDTAVYY |
| | CVRHGNFGNSYVSWFAYWGQGTLVTVSS (SEQ ID NO: 2350) |
| VL | DIQMTQSPSSLSASVGDRVTITCRSSTGAVTTSNYANWVQQKPGKAPKGL |
| | IGGTNKRAPGVPARFSGSGSGTDFTLTISSLQPEDFATYYCALWYSNLWV |
| | FGQGTKVEIK (SEQ ID NO: 2351) |

TABLE 4

| | Template Ab1 | Template Ab2 |
|---|---|---|
| HCDR1 Library | 2000 | 2000 |
| HCDR2 Library | 2000 | 2000 |
| HCDR3 Library | 2000 | 2000 |
| LCDR1 Library | 2000 | 2000 |
| LCDR2 Library | 1000 | 1000 |
| LCDR3 Library | 2000 | 2000 |

Cell Sorting of Mammalian Display Libraries. CDR libraries were transfected into a mammalian display system for cell sorting. Libraries were sorted by antibody expression (APC-conjugated anti-human Fc secondary) and binding (FITC-conjugated streptavidin-CD3ed/CD3eg complex). Gates were drawn to collect the antibody-expressing populations over a range of binding levels. Sorted cells were pelleted down and stored at −80° C. until further processing.

NGS Sequencing and Analysis. Sorted cells were sequenced by NGS to identify clone hits. This involved cell lysis, reverse transcription into cDNA, and PCR amplification with primers appropriate for sequencing the DNA library pools on an Illumina MiSeq. Sequences were rank ordered by enrichment of clones in the binding bins compared to non-binding bins in the cell sorting. Enrichment scores were calculated as the frequency of a given sequence in a binding bin divided by its frequency in a non-binding bin. Clones with enrichment scores of 10 or higher were subject to further testing within the capacity of the screening system (up to 10 sequences were tested from each CDR diversity library).

Immunizations. Engineered epitopes were chemically conjugated to keyhole limpet hemocyanin or ferritin for immunizations (no adjuvants were used). Every week, BALB/c mice were immunized with engineered epitopes one day, and isolated human T-cells on a separate day. After 6-8 weeks of alternating between engineered epitope and T-cell immunizations, mice were immunized with a final boost containing a cocktail of engineered epitopes and human T-cells.

Hybridoma Generation. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

ELISA CD3 Binding Screen. Antibodies were screened for binding of CD3 heterodimers by ELISA. Biotinylated CD3 epsilon+delta and CD3 epsilon+gamma were coated together in a 1:1 ratio onto streptavidin coated polystyrene assay plates for 1 hour. Plates were washed three times with PBST+1% BSA, and antibodies were incubated for 1 hour, followed by three more washes with PBST+1% BSA. Antibodies were then labeled with a species appropriate secondary antibody conjugated to horse radish peroxidase, and incubated for 1 hour. Plates were then washed three more times with PBST+1% BSA, and TMB substrate was added. After a 10 minute incubation, stop solution was added and absorbance was read out by a plate reader.

T Cell Binding Screen with Human PBMCs. Antibodies were incubated with Human PBMCs for 1 hour, washed once with DPBS+1% FBS, then labeled with secondary antibodies including anti human CD4, anti-human CD8, and a species appropriate anti-Fc for 1 hour. Cells were then washed twice more and measured by flow cytometry. CD3 binding of antibodies is measured on T cells that are CD4 or CD8 positive within the human PBMC mixture.

Label-Free CD3 Binding Kinetics as Measured by SPR. Binding kinetics of antibodies to CD3 heterodimers was carried out on a Carterra LSA. Antibodies were captured on an anti-Human Fc functionalized HC200M chip for 25 min, followed by 3 buffer injections to establish a blank, followed by 7 injections of a 1:3 dilution series of CD3 ordered by increasing concentration. Injection cycles consisted of a 60 second baseline, followed by a 4 minute association, followed by a 6 minute dissociation. Kinetic parameters were then fit to the data using the LSA Kinetics software.

In-Vitro Mammalian Display Library Construction from Mouse B-Cell Repertoires. Splenocytes were collected from mice after immunization, lysed in TRIzol reagent, and RNA collected by Phenol:Chloroform extraction. After reverse transcription, variable heavy and variable light chains were amplified using a primer set designed based on the IMGT database of murine germline sequences (see Schaefer et al., Antibody Engineering Vol. 1 21:42 (2010), relevant portions incorporated by reference) that add adaptors containing IIs restriction enzyme sites that allow assembly into a transmembrane bound mammalian expression plasmid in scFv format. Assembled plasmids are transformed into bacteria and collected after an overnight culture under selection conditions. Recovered plasmids are then transiently transfected into Expi293F expression cell lines to generate a mammalian display library for subsequent sorting.

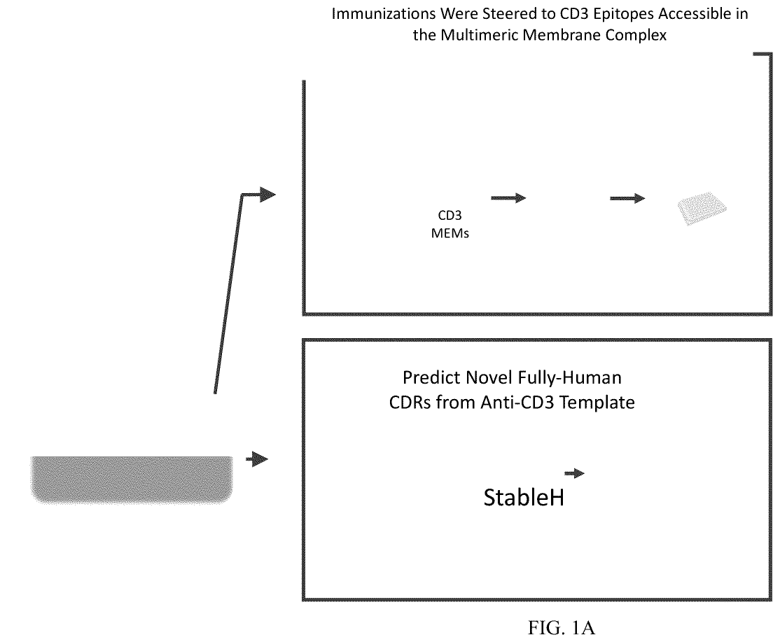

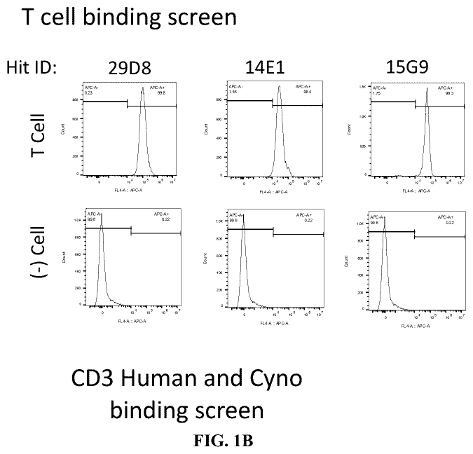

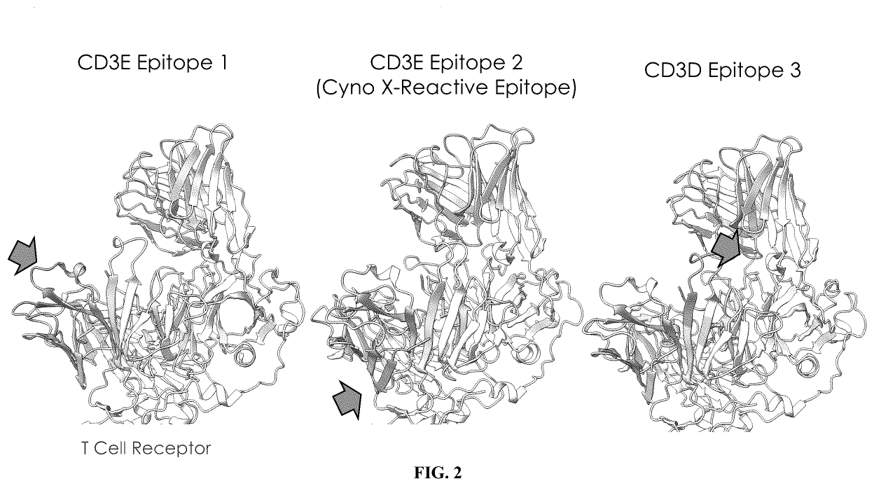

FIGS. 1A and 1B illustrates the two paths to anti-CD3 specific antibody discovery (FIG. 1A) and the method of screening (FIG. 1B). FIG. 2 shows the engineered epitopes steered immunization to three CD3E and CD3D epitopes.

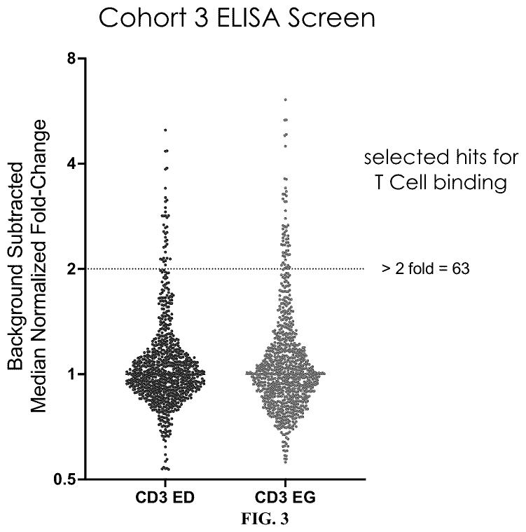

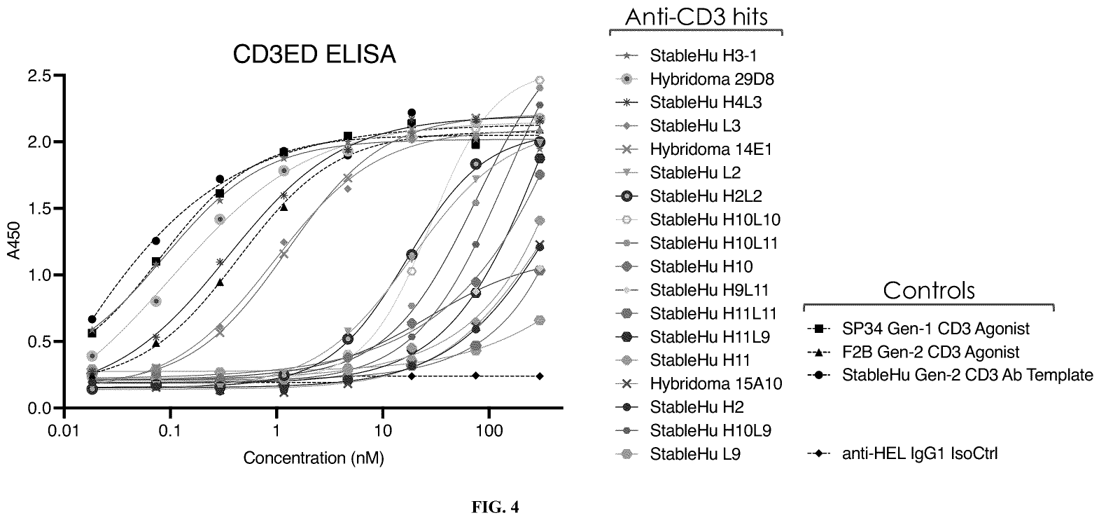

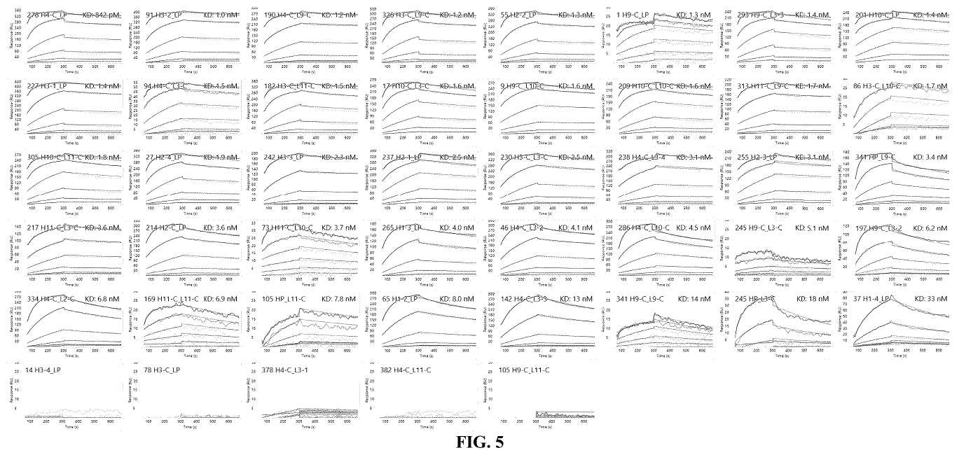

FIG. 3 shows the immunized cohorts CD3 hybridoma primary screens. FIG. 4 is a graph that shows epitope-steered immunization and StableHu generated CD3 hits across a broad binding range. FIG. 5 shows that the StableHu generated a panel of CD3 Hits with a range of binding affinities.

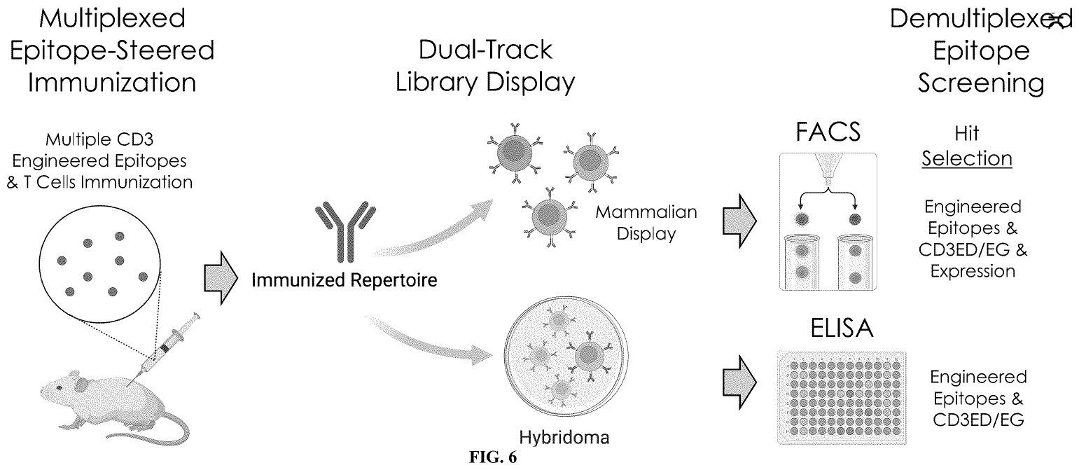

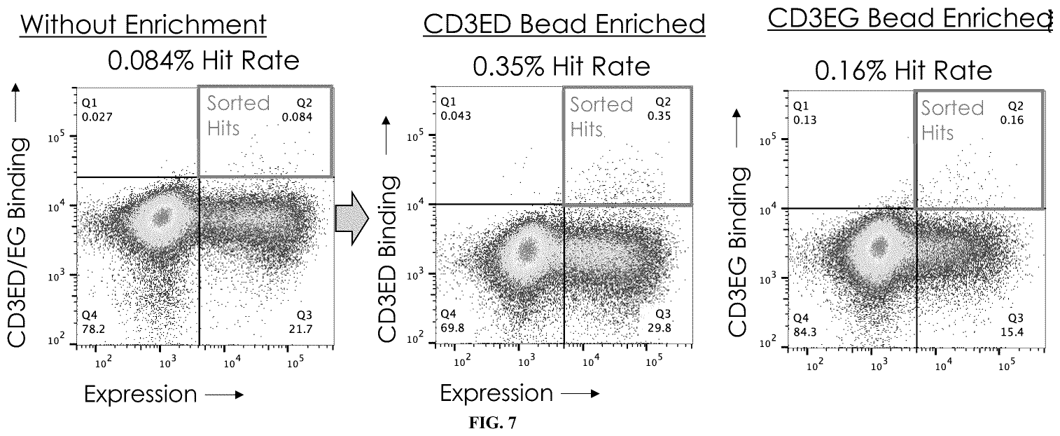

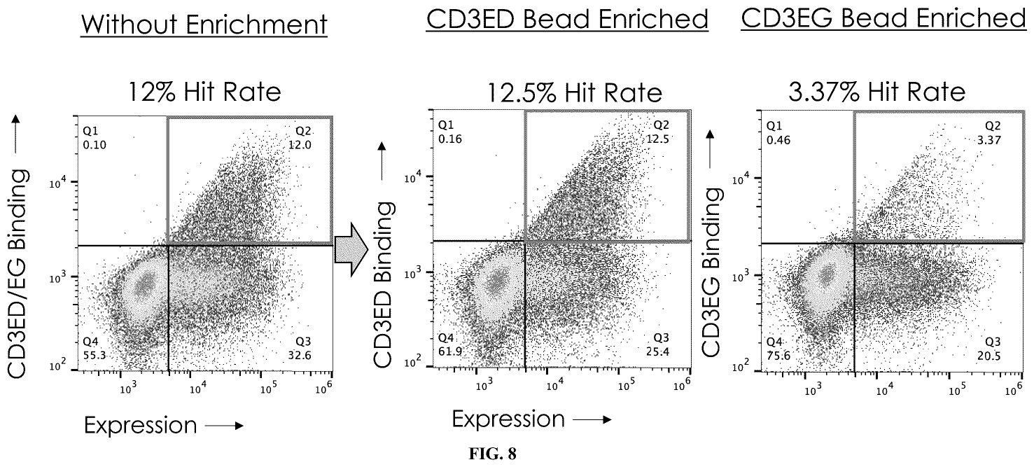

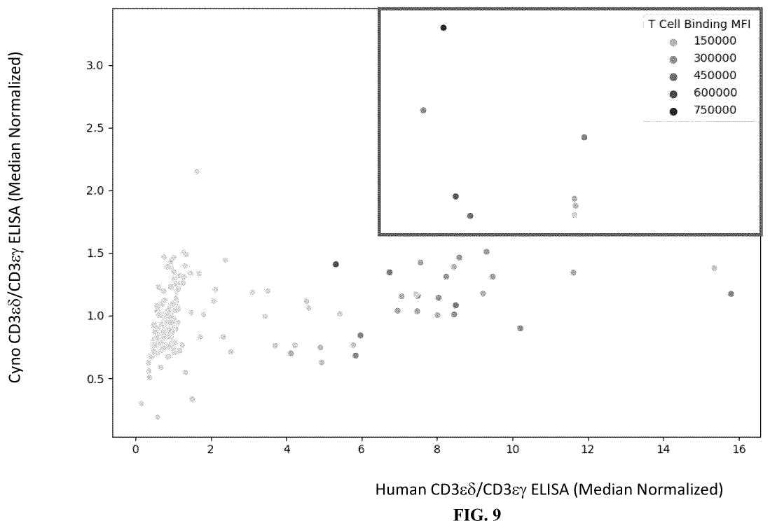

FIG. 6 shows that the immunized CD3 repertoire was cloned into mammalian display for demultiplexed screening. FIG. 7 shows the results from the first round of mammalian display screening of multi-epitope immunized CD3 repertoire. FIG. 8 shows the results from the second round of immunized CD3 mammalian display repertoire that is significantly enriched to binders. FIG. 9 shows that the ELISA screening identifies Hu-Cyno cross-reactive T-Cell binders.

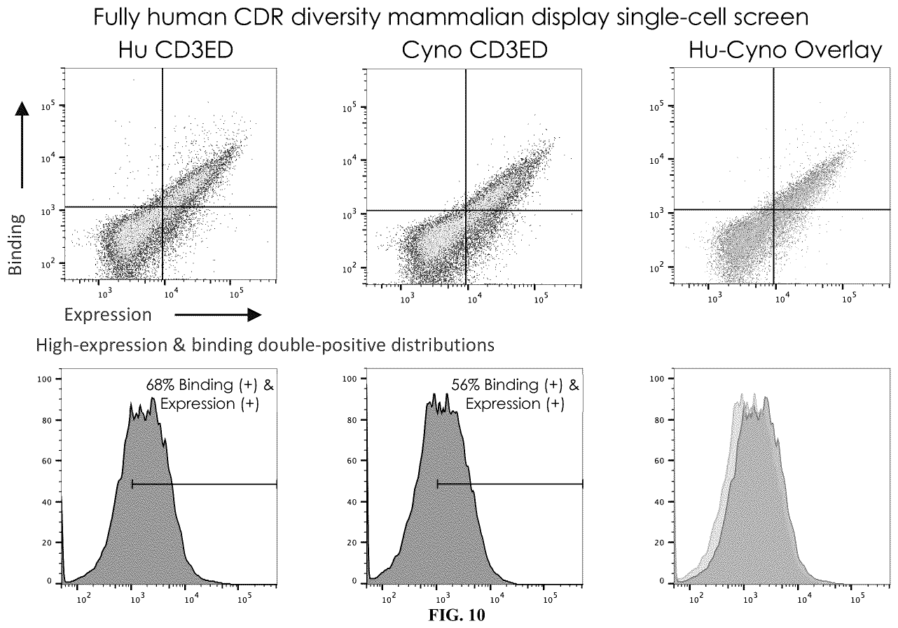

FIG. 10 shows that StableHu generates a library of Hu-Cyno cross-reactivity from anti-CD3 murine CDR template. Significant overlap in Hu-Cyno library binding distributions, ˜50-fold binding range for 86 fully human CDR hits in-testing.

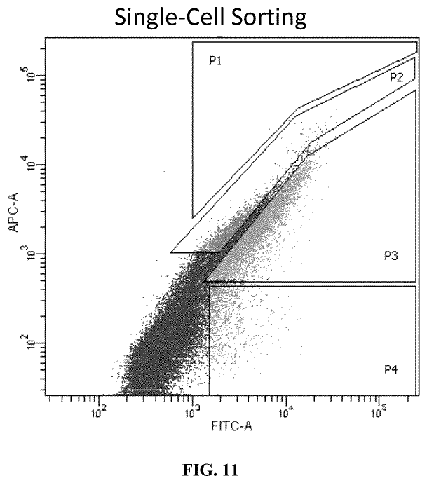

FIG. 11 shows cell sorting identifies range of Hu-Cyno Cross Reactive CD3 binders, with sequences rank-ordered from bin-weight analysis.

Lengthy table referenced here
US20240327515A1-20241003-T00001
Please refer to the end of the specification for access instructions.

Lengthy table referenced here
US20240327515A1-20241003-T00002
Please refer to the end of the specification for access instructions.

Lengthy table referenced here
US20240327515A1-20241003-T00003
Please refer to the end of the specification for access instructions.

Lengthy table referenced here
US20240327515A1-20241003-T00004
Please refer to the end of the specification for access instructions.

Lengthy table referenced here
US20240327515A1-20241003-T00005
Please refer to the end of the specification for access instructions.

Lengthy table referenced here
US20240327515A1-20241003-T00006
Please refer to the end of the specification for access instructions.

Lengthy table referenced here
US20240327515A1-20241003-T00007
Please refer to the end of the specification for access instructions.

Lengthy table referenced here
US20240327515A1-20241003-T00008
Please refer to the end of the specification for access instructions.

Lengthy table referenced here
US20240327515A1-20241003-T00009
Please refer to the end of the specification for access instructions.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%. Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

LENGTHY TABLES
The patent application contains a lengthy table section. A copy of the table is available in electronic form from the USPTO web site (https://seqdata.uspto.gov/?pageRequest=docDetail&DocID=US20240327515A1). An electronic copy of the table will also be available from the USPTO upon request and payment of the fee set forth in 37 CFR 1.19(b)(3).

What is claimed is:

1. An anti-CD3 antibody, wherein the antibody comprises:
a. a heavy chain variable domain (VH) complementarity determining region 1 (CDR1) comprising the amino acid sequence of any one of the following SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, 801, 811, 821, 831, 841, 851, 861, 871, 881, 891, 901, 911, 921, 931, 941, 951, 961, 971, 981, 991, 1001, 1011, 1021, 1031, 1041, 1051, 1061, 1071, 1081, 1091, 1101, 1111, 1121, 1131, 1141, 1151, 1161, 1171, 1181, 1191, 1201, 1211, 1221, 1231, 1241, 1251, 1261, 1271, 1281, 1291, 1301, 1311, 1321, 1331, 1341, 1351, 1361, 1371, 1381, 1391, 1401, 1411, 1421, 1431, 1441, 1451, 1461, 1471, 1481, 1491, 1501, 1511, 1521, 1531, 1541, 1551, 1561, 1571, 1581, 1591, 1601, 1611, 1621, 1631, 1641, 1651, 1661, 1671, 1681, 1691, 1701, 1711, 1721, 1731, 1741, 1751, 1761, 1771, 1781, 1791, 1801, 1811, 1821, 1831, 1841, 1851, 1861, 1871, 1881, 1891, 1901, 1911, 1916, 1921, 1926, 1931, 1936, 1941, 1946, 1951, 1956, 1961, 1966, 1971, 1976, 1981, 1986, 1991, 1996, 2001, 2006, 2011, 2016, 2021, 2026, 2031, 2036, 2041, 2046, 2051, 2056, 2061, 2066, 2071, 2076, 2081, 2086, 2091, 2096, 2101, 2106, 2111, 2116, 2121, 2126, 2131, 2136, 2141, 2146, 2151, 2156, 2161, 2166, 2171, 2176, 2181, or 2186; and
b. a VH CDR2 comprising the amino acid sequence of any one of the following SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, 792, 802, 812, 822, 832, 842, 852, 862, 872, 882, 892, 902, 912, 922, 932, 942, 952, 962, 972, 982, 992, 1002, 1012, 1022, 1032, 1042, 1052, 1062, 1072, 1082, 1092, 1102, 1112, 1122, 1132, 1142, 1152, 1162, 1172, 1182, 1192, 1202, 1212, 1222, 1232, 1242, 1252, 1262, 1272, 1282, 1292, 1302, 1312, 1322, 1332, 1342, 1352, 1362, 1372, 1382, 1392, 1402, 1412, 1422, 1432, 1442, 1452, 1462, 1472, 1482, 1492, 1502, 1512, 1522, 1532, 1542, 1552, 1562, 1572, 1582, 1592, 1602, 1612, 1622, 1632, 1642, 1652, 1662, 1672, 1682, 1692, 1702, 1712, 1722, 1732, 1742, 1752, 1762, 1772, 1782, 1792, 1802, 1812, 1822, 1832, 1842, 1852, 1862, 1872, 1882, 1892, 1902, 1912, 1917, 1922, 1927, 1932, 1937, 1942, 1947, 1952, 1957, 1962, 1967, 1972, 1977, 1982, 1987, 1992, 1997, 2002, 2007, 2012, 2017, 2022, 2027, 2032, 2037, 2042, 2047, 2052, 2057, 2062, 2067, 2072, 2077, 2082, 2087, 2092, 2097, 2102, 2107, 2112, 2117, 2122, 2127, 2132, 2137, 2142, 2147, 2152, 2157, 2162, 2167, 2172, 2177, 2182, or 2187; and
c. a VH CDR3 comprising the amino acid sequence of any one of the following SEQ ID NOs: 3, 13, 23, 33, 43, 53, 63, 73, 83, 93, 103, 113, 123, 133, 143, 153, 163, 173, 183, 193, 203, 213, 223, 233, 243, 253, 263, 273, 283, 293, 303, 313, 323, 333, 343, 353, 363, 373, 383, 393, 403, 413, 423, 433, 443, 453, 463, 473, 483, 493, 503, 513, 523, 533, 543, 553, 563, 573, 583, 593, 603, 613, 623, 633, 643, 653, 663, 673, 683, 693, 703, 713, 723, 733, 743, 753, 763, 773, 783, 793, 803, 813, 823, 833, 843, 853, 863, 873, 883, 893, 903, 913, 923, 933, 943, 953, 963, 973, 983, 993, 1003, 1013, 1023, 1033, 1043, 1053, 1063, 1073, 1083, 1093, 1103, 1113, 1123, 1133, 1143, 1153, 1163, 1173, 1183, 1193, 1203, 1213, 1223, 1233, 1243, 1253, 1263, 1273, 1283, 1293, 1303, 1313, 1323, 1333, 1343, 1353, 1363, 1373, 1383, 1393, 1403, 1413, 1423, 1433, 1443, 1453, 1463, 1473, 1483, 1493, 1503, 1513, 1523, 1533, 1543, 1553, 1563, 1573, 1583, 1593, 1603, 1613, 1623, 1633, 1643, 1653, 1663, 1673, 1683, 1693, 1703, 1713, 1723, 1733, 1743, 1753, 1763, 1773, 1783, 1793, 1803, 1813, 1823, 1833, 1843, 1853, 1863, 1873, 1883, 1893, 1903, 1913, 1918, 1923, 1928, 1933, 1938, 1943, 1948, 1953, 1958, 1963, 1968, 1973, 1978, 1983, 1988, 1993, 1998, 2003, 2008, 2013, 2018, 2023, 2028, 2033, 2038, 2043, 2048, 2053, 2058, 2063, 2068, 2073, 2078, 2083, 2088, 2093, 2098, 2103, 2108, 2113, 2118, 2123, 2128, 2133, 2138, 2143, 2148, 2153, 2158, 2163, 2168, 2173, 2178, 2183, or 2188; and
d. a light chain variable domain (VL) CDR1 comprising the amino acid sequence of any one of the following SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, 796, 806, 816, 826, 836, 846, 856, 866, 876, 886, 896, 906, 916, 926, 936, 946, 956, 966, 976, 986, 996, 1006, 1016, 1026, 1036, 1046, 1056, 1066, 1076, 1086, 1096, 1106, 1116, 1126, 1136, 1146, 1156, 1166, 1176, 1186, 1196, 1206, 1216, 1226, 1236, 1246, 1256, 1266, 1276, 1286, 1296, 1306, 1316, 1326, 1336, 1346, 1356, 1366, 1376, 1386, 1396, 1406, 1416, 1426, 1436, 1446, 1456, 1466, 1476, 1486, 1496, 1506, 1516, 1526, 1536, 1546, 1556, 1566, 1576, 1586, 1596, 1606, 1616, 1626, 1636, 1646, 1656, 1666, 1676, 1686, 1696, 1706, 1716, 1726, 1736, 1746, 1756, 1766, 1776, 1786, 1796, 1806, 1816, 1826, 1836, 1846, 1856, 1866, 1876, 1886, 1896, 1906, 2161, 2166, 2171, 2176, 2181, 2186, 2191, 2196, 2201, 2206, 2211, 2216, 2221, 2226, 2231, 2236, 2241, 2246, 2251, 2256, 2261, 2266, 2271, 2276, 2281, 2286, 2291, 2296, 2301, 2306, 2311, 2316, 2321, 2326, 2331, or 2336; and
e. a VL CDR2 comprising the amino acid sequence of any one of the following SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, 797, 807, 817, 827, 837, 847, 857, 867, 877, 887, 897, 907, 917, 927, 937, 947, 957, 967, 977, 987, 997, 1007, 1017, 1027, 1037, 1047, 1057, 1067, 1077, 1087, 1097, 1107, 1117, 1127, 1137, 1147, 1157, 1167, 1177, 1187, 1197, 1207, 1217, 1227, 1237, 1247, 1257, 1267, 1277, 1287, 1297, 1307, 1317, 1327, 1337, 1347, 1357, 1367, 1377, 1387, 1397, 1407, 1417, 1427, 1437, 1447, 1457, 1467, 1477, 1487, 1497, 1507, 1517, 1527, 1537, 1547, 1557, 1567, 1577, 1587, 1597, 1607, 1617, 1627, 1637, 1647, 1657, 1667, 1677, 1687, 1697, 1707, 1717, 1727, 1737, 1747, 1757, 1767, 1777, 1787, 1797, 1807, 1817, 1827, 1837, 1847, 1857, 1867, 1877, 1887, 1897, 1907, 2162, 2167, 2172, 2177, 2182, 2187, 2192, 2197, 2202, 2207, 2212, 2217, 2222, 2227, 2232, 2237, 2242, 2247, 2252, 2257, 2262, 2267, 2272, 2277, 2282, 2287, 2292, 2297, 2302, 2307, 2312, 2317, 2322, 2327, 2332, or 2337; and
f. a VL CDR3 comprising the amino acid sequence of any one of the following SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, 528, 538, 548, 558, 568, 578, 588, 598, 608, 618, 628, 638, 648, 658, 668, 678, 688, 698, 708, 718, 728, 738, 748, 758, 768, 778, 788, 798, 808, 818, 828, 838, 848, 858, 868, 878, 888, 898, 908, 918, 928, 938, 948, 958, 968, 978, 988, 998, 1008, 1018, 1028, 1038, 1048, 1058, 1068, 1078, 1088, 1098, 1108, 1118, 1128, 1138, 1148, 1158, 1168, 1178, 1188, 1198, 1208, 1218, 1228, 1238, 1248, 1258, 1268, 1278, 1288, 1298, 1308, 1318, 1328, 1338, 1348, 1358, 1368, 1378, 1388, 1398, 1408, 1418, 1428, 1438, 1448, 1458, 1468, 1478, 1488, 1498, 1508, 1518, 1528, 1538, 1548, 1558, 1568, 1578, 1588, 1598, 1608, 1618, 1628, 1638, 1648, 1658, 1668, 1678, 1688, 1698, 1708, 1718, 1728, 1738, 1748, 1758, 1768, 1778, 1788, 1798, 1808, 1818, 1828, 1838, 1848, 1858, 1868, 1878, 1888, 1898, 1908, 2163, 2168, 2173, 2178, 2183, 2188, 2193, 2198, 2203, 2208, 2213, 2218, 2223, 2228, 2233, 2238, 2243, 2248, 2253, 2258, 2263, 2268, 2273, 2278, 2283, 2288, 2293, 2298, 2303, 2308, 2313, 2318, 2323, 2328, 2333, or 2338.

2. The antibody of claim 1, wherein the antibody comprises:
a. a heavy chain comprising the amino acid sequence of any one of the following SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714, 724, 734, 744, 754, 764, 774, 784, 794, 804, 814, 824, 834, 844, 854, 864, 874, 884, 894, 904, 914, 924, 934, 944, 954, 964, 974, 984, 994, 1004, 1014, 1024, 1034, 1044, 1054, 1064, 1074, 1084, 1094, 1104, 1114, 1124, 1134, 1144, 1154, 1164, 1174, 1184, 1194, 1204, 1214, 1224, 1234, 1244, 1254, 1264, 1274, 1284, 1294, 1304, 1314, 1324, 1334, 1344, 1354, 1364, 1374, 1384, 1394, 1404, 1414, 1424, 1434, 1444, 1454, 1464, 1474, 1484, 1494, 1504, 1514, 1524, 1534, 1544, 1554, 1564, 1574, 1584, 1594, 1604, 1614, 1624, 1634, 1644, 1654, 1664, 1674, 1684, 1694, 1704, 1714, 1724, 1734, 1744, 1754, 1764, 1774, 1784, 1794, 1804, 1814, 1824, 1834, 1844, 1854, 1864, 1874, 1884, 1894, 1904, 1914, 1919, 1924, 1929, 1934, 1939, 1944, 1949, 1954, 1959, 1964, 1969, 1974, 1979, 1984, 1989, 1994, 1999, 2004, 2009, 2014, 2019, 2024, 2029, 2034, 2039, 2044, 2049, 2054, 2059, 2064, 2069, 2074, 2079, 2084, 2089, 2094, 2099, 2104, 2109, 2114, 2119, 2124, 2129, 2134, 2139, 2144, 2149, 2154, 2159, 2164, 2169, 2174, 2179, 2184, or 2189, and
b. a light chain comprising the amino acid sequence of any one of the following SEQ ID NOs: 9, 19, 29, 39, 49, 59, 69, 79, 89, 99, 109, 119, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 509, 519, 529, 539, 549, 559, 569, 579, 589, 599, 609, 619, 629, 639, 649, 659, 669, 679, 689, 699, 709, 719, 729, 739, 749, 759, 769, 779, 789, 799, 809, 819, 829, 839, 849, 859, 869, 879, 889, 899, 909, 919, 929, 939, 949, 959, 969, 979, 989, 999, 1009, 1019, 1029, 1039, 1049, 1059, 1069, 1079, 1089, 1099, 1109, 1119, 1129, 1139, 1149, 1159, 1169, 1179, 1189, 1199, 1209, 1219, 1229, 1239, 1249, 1259, 1269, 1279, 1289, 1299, 1309, 1319, 1329, 1339, 1349, 1359, 1369, 1379, 1389, 1399, 1409, 1419, 1429, 1439, 1449, 1459, 1469, 1479, 1489, 1499, 1509, 1519, 1529, 1539, 1549, 1559, 1569, 1579, 1589, 1599, 1609, 1619, 1629, 1639, 1649, 1659, 1669, 1679, 1689, 1699, 1709, 1719, 1729, 1739, 1749, 1759, 1769, 1779, 1789, 1799, 1809, 1819, 1829, 1839, 1849, 1859, 1869, 1879, 1889, 1899, 1909, 2164, 2169, 2174, 2179, 2184, 2189, 2194, 2199, 2204, 2209, 2214, 2219, 2224, 2229, 2234, 2239, 2244, 2249, 2254, 2259, 2264, 2269, 2274, 2279, 2284, 2289, 2294, 2299, 2304, 2309, 2314, 2319, 2324, 2329, 2334, or 2339.

3. The antibody of claim 1, wherein the antibody is encoded by a nucleic acid that comprises:
a. a VH comprising the nucleic acid sequence of any one of the following SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, 805, 815, 825, 835, 845, 855, 865, 875, 885, 895, 905, 915, 925, 935, 945, 955, 965, 975, 985, 995, 1005, 1015, 1025, 1035, 1045, 1055, 1065, 1075, 1085, 1095, 1105, 1115, 1125, 1135, 1145, 1155, 1165, 1175, 1185, 1195, 1205, 1215, 1225, 1235, 1245, 1255, 1265, 1275, 1285, 1295, 1305, 1315, 1325, 1335, 1345, 1355, 1365, 1375, 1385, 1395, 1405, 1415, 1425, 1435, 1445, 1455, 1465, 1475, 1485, 1495, 1505, 1515, 1525, 1535, 1545, 1555, 1565, 1575, 1585, 1595, 1605, 1615, 1625, 1635, 1645, 1655, 1665, 1675, 1685, 1695, 1705, 1715, 1725, 1735, 1745, 1755, 1765, 1775, 1785, 1795, 1805, 1815, 1825, 1835, 1845, 1855, 1865, 1875, 1885, 1895, 1905, 1915, 1920, 1925, 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, or 2190, and
b. a VL comprising the nucleic acid sequence of any one of the following SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, or 2340.

4. The antibody of claim 1, wherein the antibody comprises:
a heavy chain variable domain (VH) comprising complementarity determining regions CDR1 to CDR3, comprising the amino acid sequence triplets: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; 491, 492, 493; 501, 502, 503; 511, 512, 513; 521, 522, 523; 531, 532, 533; 541, 542, 543; 551, 552, 553; 561, 562, 563; 571, 572, 573; 581, 582, 583; 591, 592, 593; 601, 602, 603; 611, 612, 613; 621, 622, 623; 631, 632, 633; 641, 642, 643; 651, 652, 653; 661, 662, 663; 671, 672, 673; 681, 682, 683; 691, 692, 693; 701, 702, 703; 711, 712, 713; 721, 722, 723; 731, 732, 733; 741, 742, 743; 751, 752, 753; 761, 762, 763; 771, 772, 773; 781, 782, 783; 791, 792, 793; 801, 802, 803; 811, 812, 813; 821, 822, 823; 831, 832, 833; 841, 842, 843; 851, 852, 853; 861, 862, 863; 871, 872, 873; 881, 882, 883; 891, 892, 893; 901, 902, 903; 911, 912, 913; 921, 922, 923; 931, 932, 933; 941, 942, 943; 951, 952, 953; 961, 962, 963; 971, 972, 973; 981, 982, 983; 991, 992, 993; 1001, 1002, 1003; 1011, 1012, 1013; 1021, 1022, 1023; 1031, 1032, 1033; 1041, 1042, 1043; 1051, 1052, 1053; 1061, 1062, 1063; 1071, 1072, 1073; 1081, 1082, 1083; 1091, 1092, 1093; 1101, 1102, 1103; 1111, 1112, 1113; 1121, 1122, 1123; 1131, 1132, 1133; 1141, 1142, 1143; 1151, 1152, 1153; 1161, 1162, 1163; 1171, 1172, 1173; 1181, 1182, 1183; 1191, 1192, 1193; 1201, 1202, 1203; 1211, 1212, 1213; 1221, 1222, 1223; 1231, 1232, 1233; 1241, 1242, 1243; 1251, 1252, 1253; 1261, 1262, 1263; 1271, 1272, 1273; 1281, 1282, 1283; 1291, 1292, 1293; 1301, 1302, 1303; 1311, 1312, 1313; 1321, 1322, 1323; 1331, 1332, 1333; 1341, 1342, 1343; 1351, 1352, 1353; 1361, 1362, 1363; 1371, 1372, 1373; 1381, 1382, 1383; 1391, 1392, 1393; 1401, 1402, 1403; 1411, 1412, 1413; 1421, 1422, 1423; 1431, 1432, 1433; 1441, 1442, 1443; 1451, 1452, 1453; 1461, 1462, 1463; 1471, 1472, 1473; 1481, 1482, 1483; 1491, 1492, 1493; 1501, 1502, 1503; 1511, 1512, 1513; 1521, 1522, 1523; 1531, 1532, 1533; 1541, 1542, 1543; 1551, 1552, 1553; 1561, 1562, 1563; 1571, 1572, 1573; 1581, 1582, 1583; 1591, 1592, 1593; 1601, 1602, 1603; 1611, 1612, 1613; 1621, 1622, 1623; 1631, 1632, 1633; 1641, 1642, 1643; 1651, 1652, 1653; 1661, 1662, 1663; 1671, 1672, 1673; 1681, 1682, 1683; 1691, 1692, 1693; 1701, 1702, 1703; 1711, 1712, 1713; 1721, 1722, 1723; 1731, 1732, 1733; 1741, 1742, 1743; 1751, 1752, 1753; 1761, 1762, 1763; 1771, 1772, 1773; 1781, 1782, 1783; 1791, 1792, 1793; 1801, 1802, 1803; 1811, 1812, 1813; 1821, 1822, 1823; 1831, 1832, 1833; 1841, 1842, 1843; 1851, 1852, 1853; 1861, 1862, 1863; 1871, 1872, 1873; 1881, 1882, 1883; 1891, 1892, 1893; 1901, 1902, 1903; 1911, 1912, 1913; 1921, 1922, 1923; or 1931, 1932, 1933; and
a light chain variable domain (VL) comprising complementarity determining regions CDR1 to CDR3, comprising the amino acid sequence triplets: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; 256, 257, 258; 266, 267, 268; 276, 277, 278; 286, 287, 288; 296, 297, 298; 306, 307, 308; 316, 317, 318; 326, 327, 328; 336, 337, 338; 346, 347, 348; 356, 357, 358; 366, 367, 368; 376, 377, 378; 386, 387, 388; 396, 397, 398; 406, 407, 408; 416, 417, 418; 426, 427, 428; 436, 437, 438; 446, 447, 448; 456, 457, 458; 466, 467, 468; 476, 477, 478; 486, 487, 488; 496, 497, 498; 506, 507, 508; 516, 517, 518; 526, 527, 528; 536, 537, 538; 546, 547, 548; 556, 557, 558; 566, 567, 568; 576, 577, 578; 586, 587, 588; 596, 597, 598; 606, 607, 608; 616, 617, 618; 626, 627, 628; 636, 637, 638; 646, 647, 648; 656, 657, 658; 666, 667, 668; 676, 677, 678; 686, 687, 688; 696, 697, 698; 706, 707, 708; 716, 717, 718; 726, 727, 728; 736, 737, 738; 746, 747, 748; 756, 757, 758; 766, 767, 768; 776, 777, 778; 786, 787, 788; 796, 797, 798; 806, 807, 808; 816, 817, 818; 826, 827, 828; 836, 837, 838; 846, 847, 848; 856, 857, 858; 866, 867, 868; 876, 877, 878; 886, 887, 888; 896, 897, 898; 906, 907, 908; 916, 917, 918; 926, 927, 928; 936, 937, 938; 946, 947, 948; 956, 957, 958; 966, 967, 968; 976, 977, 978; 986, 987, 988; 996, 997, 998; 1006, 1007, 1008; 1016, 1017, 1018; 1026, 1027, 1028; 1036, 1037, 1038; 1046, 1047, 1048; 1056, 1057, 1058; 1066, 1067, 1068; 1076, 1077, 1078; 1086, 1087, 1088; 1096, 1097, 1098; 1106, 1107, 1108; 1116, 1117, 1118; 1126, 1127, 1128; 1136, 1137, 1138; 1146, 1147, 1148; 1156, 1157, 1158; 1166, 1167, 1168; 1176, 1177, 1178; 1186, 1187, 1188; 1196, 1197, 1198; 1206, 1207, 1208; 1216, 1217, 1218; 1226, 1227, 1228; 1236, 1237, 1238; 1246, 1247, 1248; 1256, 1257, 1258; 1266, 1267, 1268; 1276, 1277, 1278; 1286, 1287, 1288; 1296, 1297, 1298; 1306, 1307, 1308; 1316, 1317, 1318; 1326, 1327, 1328; 1336, 1337, 1338; 1346, 1347, 1348; 1356, 1357, 1358; 1366, 1367, 1368; 1376, 1377, 1378; 1386, 1387, 1388; 1396, 1397, 1398; 1406, 1407, 1408; 1416, 1417, 1418; 1426, 1427, 1428; 1436, 1437, 1438; 1446, 1447, 1448; 1456, 1457, 1458; 1466, 1467, 1468; 1476, 1477, 1478; 1486, 1487, 1488; 1496, 1497, 1498; 1506, 1507, 1508; 1516, 1517, 1518; 1526, 1527, 1528; 1536, 1537, 1538; 1546, 1547, 1548; 1556, 1557, 1558; 1566, 1567, 1568; 1576, 1577, 1578; 1586, 1587, 1588; 1596, 1597, 1598; 1606, 1607, 1608; 1616, 1617, 1618; 1626, 1627, 1628; 1636, 1637, 1638; 1646, 1647, 1648; 1656, 1657, 1658; 1666, 1667, 1668; 1676, 1677, 1678; 1686, 1687, 1688; 1696, 1697, 1698; 1706, 1707, 1708; 1716, 1717, 1718; 1726, 1727, 1728; 1736, 1737, 1738; 1746, 1747, 1748; 1756, 1757, 1758; 1766, 1767, 1768; 1776, 1777, 1778; 1786, 1787, 1788; 1796, 1797, 1798; 1806, 1807, 1808; 1816, 1817, 1818; 1826, 1827, 1828; 1836, 1837, 1838; 1846, 1847, 1848; 1856, 1857, 1858; 1866, 1867, 1868; 1876, 1877, 1878; 1886, 1887, 1888; 1896, 1897, 1898; 1906, 1907, 1908; 1916, 1917, 1918; 1926, 1927, 1928; or 1936, 1937, 1938.

5. The antibody of claim 1, wherein the antibody comprises:
a heavy chain variable domain (VH) comprising complementarity determining regions CDR1 to CDR3, comprising the amino acid sequence triplets: 1941, 1942, 1943; 1946, 1947, 1948; 1951, 1952, 1953; 1956, 1957, 1958; 1961, 1962, 1963; 1966, 1967, 1968; 1971, 1972, 1973; 1976, 1977, 1978; 1981, 1982, 1983; 1986, 1987, 1988; 1991, 1992, 1993; 1996, 1997, 1998; 2001, 2002, 2003; 2006, 2007, 2008; 2011, 2012, 2013; 2016, 2017, 2018; 2021, 2022, 2023; 2026, 2027, 2028; 2031, 2032, 2033; 2036, 2037, 2038; 2041, 2042, 2043; 2046, 2047, 2048; 2051, 2052, 2053; 2056, 2057, 2058; 2061, 2062, 2063; 2066, 2067, 2068; 2071, 2072, 2073; 2076, 2077, 2078; 2081, 2082, 2083; 2086, 2087, 2088; 2091, 2092, 2093; 2096, 2097, 2098; 2101, 2102, 2103; 2106, 2107, 2108; 2111, 2112, 2113; 2116, 2117, 2118; 2121, 2122, 2123; 2126, 2127, 2128; 2131, 2132, 2133; 2136, 2137, 2138; 2141, 2142, 2143; 2146, 2147, 2148; 2151, 2152, 2153; 2156, 2157, 2158; 2161, 2162, 2163; 2166, 2167, 2168; 2171, 2172, 2173; 2176, 2177, 2178; 2181, 2182, 2183; or 2186, 2187, 2188; and
a light chain variable domain (VL) comprising complementarity determining regions CDR1 to CDR3, comprising the amino acid sequence triplets: 2191, 2192, 2193; 2196, 2197, 2198; 2201, 2202, 2203; 2206, 2207, 2208; 2211, 2212, 2213; 2216, 2217, 2218; 2221, 2222, 2223; 2226, 2227, 2228; 2231, 2232, 2233; 2236, 2237, 2238; 2241, 2242, 2243; 2246, 2247, 2248; 2251, 2252, 2253; 2256, 2257, 2258; 2261, 2262, 2263; 2266, 2267, 2268; 2271, 2272, 2273; 2276, 2277, 2278; 2281, 2282, 2283; 2286, 2287, 2288; 2291, 2292, 2293; 2296, 2297, 2298; 2301, 2302, 2303; 2306, 2307, 2308; 2311, 2312, 2313; 2316, 2317, 2318; 2321, 2322, 2323; 2326, 2327, 2328; 2331, 2332, 2333; or 2336, 2337, 2338.

6. The antibody of claim 1, wherein the antibody is a monoclonal, bispecific, multivalent, multi-specific, diabody, chimeric, scFv antibody, or fragments thereof.

7. The antibody of claim 1, wherein the antibody is a full-length antibody.

8. The antibody of claim 1, wherein the antibody is an antibody fragment.

9. The antibody fragment of claim 8, wherein the antibody is fused to an Fc domain of any one of the following: human IgG1, human IgG2, human IgG3, and human IgG4.

10. The antibody fragment of claim 9, wherein the Fc domain is a wild-type, variant, or truncated Fc domain.

11. The antibody of claim 1, further comprising a peptide linker between a VH2 domain and the CH1 domain.

12. A method of treating a disease in a subject in need thereof, comprising administering to the subject a therapeutically effective amount of the antibody of claim 1.

13. The method of claim 12, wherein the disease is an autoimmune disease, an inflammatory disease, or a cancer.

14. The method of claim 12, wherein the subject is human.

15. A nucleic acid encoding an anti-CD3 antibody, wherein the nucleic acid comprises:
a VH comprising the nucleic acid sequence of any one of the following SEQ ID NOS: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, 805, 815, 825, 835, 845, 855, 865, 875, 885, 895, 905, 915, 925, 935, 945, 955, 965, 975, 985, 995, 1005, 1015, 1025, 1035, 1045, 1055, 1065, 1075, 1085, 1095, 1105, 1115, 1125, 1135, 1145, 1155, 1165, 1175, 1185, 1195, 1205, 1215, 1225, 1235, 1245, 1255, 1265, 1275, 1285, 1295, 1305, 1315, 1325, 1335, 1345, 1355, 1365, 1375, 1385, 1395, 1405, 1415, 1425, 1435, 1445, 1455, 1465, 1475, 1485, 1495, 1505, 1515, 1525, 1535, 1545, 1555, 1565, 1575, 1585, 1595, 1605, 1615, 1625, 1635, 1645, 1655, 1665, 1675, 1685, 1695, 1705, 1715, 1725, 1735, 1745, 1755, 1765, 1775, 1785, 1795, 1805, 1815, 1825, 1835, 1845, 1855, 1865, 1875, 1885, 1895, 1905, 1915, 1920, 1925, 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, or 2190, and
a VL comprising the nucleic acid sequence of any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, or 2340.

16. An expression vector comprising a nucleic acid encoding at least one of a nucleic acids encoding the antibody of claim 15.

17. A host cell comprising the vector of claim 16.

18. A method of making an anti-CD3 antibody comprising:
expressing in a cell a heavy chain variable domain (VH) comprising complementarity determining regions CDR1 to CDR3, comprising the amino acid sequence triplets: 1, 2, 3; 11, 12, 13; 21, 22, 23; 31, 32, 33; 41, 42, 43; 51, 52, 53; 61, 62, 63; 71, 72, 73; 81, 82, 83; 91, 92, 93; 101, 102, 103; 111, 112, 113; 121, 122, 123; 131, 132, 133; 141, 142, 143; 151, 152, 153; 161, 162, 163; 171, 172, 173; 181, 182, 183; 191, 192, 193; 201, 202, 203; 211, 212, 213; 221, 222, 223; 231, 232, 233; 241, 242, 243; 251, 252, 253; 261, 262, 263; 271, 272, 273; 281, 282, 283; 291, 292, 293; 301, 302, 303; 311, 312, 313; 321, 322, 323; 331, 332, 333; 341, 342, 343; 351, 352, 353; 361, 362, 363; 371, 372, 373; 381, 382, 383; 391, 392, 393; 401, 402, 403; 411, 412, 413; 421, 422, 423; 431, 432, 433; 441, 442, 443; 451, 452, 453; 461, 462, 463; 471, 472, 473; 481, 482, 483; 491, 492, 493; 501, 502, 503; 511, 512, 513; 521, 522, 523; 531, 532, 533; 541, 542, 543; 551, 552, 553; 561, 562, 563; 571, 572, 573; 581, 582, 583; 591, 592, 593; 601, 602, 603; 611, 612, 613; 621, 622, 623; 631, 632, 633; 641, 642, 643; 651, 652, 653; 661, 662, 663; 671, 672, 673; 681, 682, 683; 691, 692, 693; 701, 702, 703; 711, 712, 713; 721, 722, 723; 731, 732, 733; 741, 742, 743; 751, 752, 753; 761, 762, 763; 771, 772, 773; 781, 782, 783; 791, 792, 793; 801, 802, 803; 811, 812, 813; 821, 822, 823; 831, 832, 833; 841, 842, 843; 851, 852, 853; 861, 862, 863; 871, 872, 873; 881, 882, 883; 891, 892, 893; 901, 902, 903; 911, 912, 913; 921, 922, 923; 931, 932, 933; 941, 942, 943; 951, 952, 953; 961, 962, 963; 971, 972, 973; 981, 982, 983; 991, 992, 993; 1001, 1002, 1003; 1011, 1012, 1013; 1021, 1022, 1023; 1031, 1032, 1033; 1041, 1042, 1043; 1051, 1052, 1053; 1061, 1062, 1063; 1071, 1072, 1073; 1081, 1082, 1083; 1091, 1092, 1093; 1101, 1102, 1103; 1111, 1112, 1113; 1121, 1122, 1123; 1131, 1132, 1133; 1141, 1142, 1143; 1151, 1152, 1153; 1161, 1162, 1163; 1171, 1172, 1173; 1181, 1182, 1183; 1191, 1192, 1193; 1201, 1202, 1203; 1211, 1212, 1213; 1221, 1222, 1223; 1231, 1232, 1233; 1241, 1242, 1243; 1251, 1252, 1253; 1261, 1262, 1263; 1271, 1272, 1273; 1281, 1282, 1283; 1291, 1292, 1293; 1301, 1302, 1303; 1311, 1312, 1313; 1321, 1322, 1323; 1331, 1332, 1333; 1341, 1342, 1343; 1351, 1352, 1353; 1361, 1362, 1363; 1371, 1372, 1373; 1381, 1382, 1383; 1391, 1392, 1393; 1401, 1402, 1403; 1411, 1412, 1413; 1421, 1422, 1423; 1431, 1432, 1433; 1441, 1442, 1443; 1451, 1452, 1453; 1461, 1462, 1463; 1471, 1472, 1473; 1481, 1482, 1483; 1491, 1492, 1493; 1501, 1502, 1503; 1511, 1512, 1513; 1521, 1522, 1523; 1531, 1532, 1533; 1541, 1542, 1543; 1551, 1552, 1553; 1561, 1562, 1563; 1571, 1572, 1573; 1581, 1582, 1583; 1591, 1592, 1593; 1601, 1602, 1603; 1611, 1612, 1613; 1621, 1622, 1623; 1631, 1632, 1633; 1641, 1642, 1643; 1651, 1652, 1653; 1661, 1662, 1663; 1671, 1672, 1673; 1681, 1682, 1683; 1691, 1692, 1693; 1701, 1702, 1703; 1711, 1712, 1713; 1721, 1722, 1723; 1731, 1732, 1733; 1741, 1742, 1743; 1751, 1752, 1753; 1761, 1762, 1763; 1771, 1772, 1773; 1781, 1782, 1783; 1791, 1792, 1793; 1801, 1802, 1803; 1811, 1812, 1813; 1821, 1822, 1823; 1831, 1832, 1833; 1841, 1842, 1843; 1851, 1852, 1853; 1861, 1862, 1863; 1871, 1872, 1873; 1881, 1882, 1883; 1891, 1892, 1893; 1901, 1902, 1903; 1911, 1912, 1913; 1921, 1922, 1923; or 1931, 1932, 1933; and
a light chain variable domain (VL) comprising complementarity determining regions CDR1 to CDR3, comprising the amino acid sequence triplets: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; 256, 257, 258; 266, 267, 268; 276, 277, 278; 286, 287, 288; 296, 297, 298; 306, 307, 308; 316, 317, 318; 326, 327, 328; 336, 337, 338; 346, 347, 348; 356, 357, 358; 366, 367, 368; 376, 377, 378; 386, 387, 388; 396, 397, 398; 406, 407, 408; 416, 417, 418; 426, 427, 428; 436, 437, 438; 446, 447, 448; 456, 457, 458; 466, 467, 468; 476, 477, 478; 486, 487, 488; 496, 497, 498; 506, 507, 508; 516, 517, 518; 526, 527, 528; 536, 537, 538; 546, 547, 548; 556, 557, 558; 566, 567, 568; 576, 577, 578; 586, 587, 588; 596, 597, 598; 606, 607, 608; 616, 617, 618; 626, 627, 628; 636, 637, 638; 646, 647, 648; 656, 657, 658; 666, 667, 668; 676, 677, 678; 686, 687, 688; 696, 697, 698; 706, 707, 708; 716, 717, 718; 726, 727, 728; 736, 737, 738; 746, 747, 748; 756, 757, 758; 766, 767, 768; 776, 777, 778; 786, 787, 788; 796, 797, 798; 806, 807, 808; 816, 817, 818; 826, 827, 828; 836, 837, 838; 846, 847, 848; 856, 857, 858; 866, 867, 868; 876, 877, 878; 886, 887, 888; 896, 897, 898; 906, 907, 908; 916, 917, 918; 926, 927, 928; 936, 937, 938; 946, 947, 948; 956, 957, 958; 966, 967, 968; 976, 977, 978; 986, 987, 988; 996, 997, 998; 1006, 1007, 1008; 1016, 1017, 1018; 1026, 1027, 1028; 1036, 1037, 1038; 1046, 1047, 1048; 1056, 1057, 1058; 1066, 1067, 1068; 1076, 1077, 1078; 1086, 1087, 1088; 1096, 1097, 1098; 1106, 1107, 1108; 1116, 1117, 1118; 1126, 1127, 1128; 1136, 1137, 1138; 1146, 1147, 1148; 1156, 1157, 1158; 1166, 1167, 1168; 1176, 1177, 1178; 1186, 1187, 1188; 1196, 1197, 1198; 1206, 1207, 1208; 1216, 1217, 1218; 1226, 1227, 1228; 1236, 1237, 1238; 1246, 1247, 1248; 1256, 1257, 1258; 1266, 1267, 1268; 1276, 1277, 1278; 1286, 1287, 1288; 1296, 1297, 1298; 1306, 1307, 1308; 1316, 1317, 1318; 1326, 1327, 1328; 1336, 1337, 1338; 1346, 1347, 1348; 1356, 1357, 1358; 1366, 1367, 1368; 1376, 1377, 1378; 1386, 1387, 1388; 1396, 1397, 1398; 1406, 1407, 1408; 1416, 1417, 1418; 1426, 1427, 1428; 1436, 1437, 1438; 1446, 1447, 1448; 1456, 1457, 1458; 1466, 1467, 1468; 1476, 1477, 1478; 1486, 1487, 1488; 1496, 1497, 1498; 1506, 1507, 1508; 1516, 1517, 1518; 1526, 1527, 1528; 1536, 1537, 1538; 1546, 1547, 1548; 1556, 1557, 1558; 1566, 1567, 1568; 1576, 1577, 1578; 1586, 1587, 1588; 1596, 1597, 1598; 1606, 1607, 1608; 1616, 1617, 1618; 1626, 1627, 1628; 1636, 1637, 1638; 1646, 1647, 1648; 1656, 1657, 1658; 1666, 1667, 1668; 1676, 1677, 1678; 1686, 1687, 1688; 1696, 1697, 1698; 1706, 1707, 1708; 1716, 1717, 1718; 1726, 1727, 1728; 1736, 1737, 1738; 1746, 1747, 1748; 1756, 1757, 1758; 1766, 1767, 1768; 1776, 1777, 1778; 1786, 1787, 1788; 1796, 1797, 1798; 1806, 1807, 1808; 1816, 1817, 1818; 1826, 1827, 1828; 1836, 1837, 1838; 1846, 1847, 1848; 1856, 1857, 1858; 1866, 1867, 1868; 1876, 1877, 1878; 1886, 1887, 1888; 1896, 1897, 1898; 1906, 1907, 1908; 1916, 1917, 1918; 1926, 1927, 1928; or 1936, 1937, 1938; or
a heavy chain variable domain (VH) comprising complementarity determining regions CDR1 to CDR3, comprising the amino acid sequence triplets: 1941, 1942, 1943; 1946, 1947, 1948; 1951, 1952, 1953; 1956, 1957, 1958; 1961, 1962, 1963; 1966, 1967, 1968; 1971, 1972, 1973; 1976, 1977, 1978; 1981, 1982, 1983; 1986, 1987, 1988; 1991, 1992, 1993; 1996, 1997, 1998; 2001, 2002, 2003; 2006, 2007, 2008; 2011, 2012, 2013; 2016, 2017, 2018; 2021, 2022, 2023; 2026, 2027, 2028; 2031, 2032, 2033; 2036, 2037, 2038; 2041, 2042, 2043; 2046, 2047, 2048; 2051, 2052, 2053; 2056, 2057, 2058; 2061, 2062, 2063; 2066, 2067, 2068; 2071, 2072, 2073; 2076, 2077, 2078; 2081, 2082, 2083; 2086, 2087, 2088; 2091, 2092, 2093; 2096, 2097, 2098; 2101, 2102, 2103; 2106, 2107, 2108; 2111, 2112, 2113; 2116, 2117, 2118; 2121, 2122, 2123; 2126, 2127, 2128; 2131, 2132, 2133; 2136, 2137, 2138; 2141, 2142, 2143; 2146, 2147, 2148; 2151, 2152, 2153; 2156, 2157, 2158; 2161, 2162, 2163; 2166, 2167, 2168; 2171, 2172, 2173; 2176, 2177, 2178; 2181, 2182, 2183; or 2186, 2187, 2188; and
a light chain variable domain (VL) comprising complementarity determining regions CDR1 to CDR3, comprising the amino acid sequence triplets: 2191, 2192, 2193; 2196, 2197, 2198; 2201, 2202, 2203; 2206, 2207, 2208; 2211, 2212, 2213; 2216, 2217, 2218; 2221, 2222, 2223; 2226, 2227, 2228; 2231, 2232, 2233; 2236, 2237, 2238; 2241, 2242, 2243; 2246, 2247, 2248; 2251, 2252, 2253; 2256, 2257, 2258; 2261, 2262, 2263; 2266, 2267, 2268; 2271, 2272, 2273; 2276, 2277, 2278; 2281, 2282, 2283; 2286, 2287, 2288; 2291, 2292, 2293; 2296, 2297, 2298; 2301, 2302, 2303; 2306, 2307, 2308; 2311, 2312, 2313; 2316, 2317, 2318; 2321, 2322, 2323; 2326, 2327, 2328; 2331, 2332, 2333; or 2336, 2337, 2338.

19. The method of claim 18, wherein the antibody is at least one of:
a monoclonal, bispecific, multivalent, multi-specific, diabody, chimeric, scFv antibody;
a fragment thereof;
a full-length antibody, is an antibody fragment,
an Fc domain of any one of the following: human IgG1, human IgG2, human IgG3,
and human IgG4; or
Fc domain is a wild-type, variant, or truncated Fc domain.

20. The method of claim 18, further comprising a peptide linker between a VH2 domain and the CH1 domain.
